(19) United States
(12) Patent Application Publication
(10) Pub. No.: US 2010/0015128 A1
(43) Pub. Date: Jan. 21, 2010

(54) GRP78 as a Predictor of Responsiveness to Therapeutic Agents

(75) Inventors: Amy S. Lee, San Marino, CA (US); Richard Cote, Los Angeles, CA (US)

Correspondence Address: FISH & RICHARDSON P.C., P.O BOX 1022, Minneapolis, MN, 55440-1022, US

(73) Assignee: UNIVERSITY OF SOUTHERN CALIFORNIA, Los Angeles, CA (US)

(21) Appl. No.: 12/444,076

(22) PCT Filed: Aug. 15, 2007
(86) PCT No.: PCT/US07/75960

§ 371 Date: May 26, 2009

Related U.S. Application Data
(60) Provisional application No. 60/828,003, filed on Oct. 3, 2006.
(60) Provisional application No. 60/898,005, filed on Jan. 12, 2007.

(51) Int. Cl. A61K 39/395; C12Q 1/68; G01N 33/48; A61K 31/7088; A61K 31/435; A61K 31/337; A61K 31/352

(52) U.S. Cl. 424/130.1; 435/6; 435/7.92; 514/44 R; 514/44 A; 514/284; 514/449; 514/456

(57) ABSTRACT

The present application provides methods for detecting, diagnosing, monitoring, predicting responsiveness to therapeutic agents and staging cancer. The present application provides methods and compositions useful for suppression of GRP78 expression or activity in cancer cells to overcome resistance of certain therapeutic agents. The present application also provides methods for predicting whether a subject with cancer is at risk for developing resistance to certain therapeutic agents.

### CROSS REFERENCE TO RELATED APPLICATIONS

The present application claims priority to U.S. Ser. No. 60/828,003 filed Oct. 3, 2006 and U.S. Ser. No. 60/898,005 filed Jan. 12, 2007, which are incorporated by reference herein in their entireties.

### STATEMENT REGARDING FEDERALLY FUNDED RESEARCH

This invention was made with government support under Grant Nos. 5 R01 CA 027607-26 and i R R01 CA 111700-01 A1 awarded by the NIH/NCI, NIH Grant No. CA111700 and Department of Defense Grant No. W81XWH-05-1-0440. The government may have certain rights in the invention.

### BACKGROUND

The identification of cancer biomarkers opens the possibility for early detection, better monitoring of tumor progression, and even targeted therapy. Classical approaches to cancer biomarker identification involved immunizing animals with tumor cells and then screening for antibodies that recognize a cell-specific antigen (Bast, et al., N. Engl. J. Med. 309:883 73 (1983)). Recently, tumor mRNA has been compared with normal tissue mRNA in an attempt to identify up-regulated genes in cancer tissue using cDNA microarrays (Mok, et al, J. Nat'l Cancer Inst. 93:1458 64.3 (2001); Kim, et al., J. Am. Med. Assoc. 289:1671 804 5 (2002)).

The estrogen receptor is a key regulator and therapeutic target in breast cancer etiology and progression. Endocrine therapy, which blocks the estrogen receptor signaling pathways, is one of the most important systemic therapies in breast cancer treatment (Osborne and Schiff, J Clin Oncol 2005, 23:1616-22). Antiestrogens such as tamoxifen have been widely used as adjuvant therapy for women with estrogen receptor positive breast carcinoma because of its effectiveness and low toxicities compared with systemic chemotherapy. Fulvestrant (Faslodex), a estrogen receptor antagonist in clinical use in metastatic hormone receptor positive breast cancer, has no agonist activity and causes degradation of the estrogen receptor, thus eliminating estrogen-sensitive gene transcription. Third generation aromatase inhibitors (e.g., anastozole, letrozole and exemestane), which block the conversion of adrenally derived androgens to estrogen in postmenopausal women, provide better efficacy and tolerability. Despite these advances, de novo or acquired resistance is frequently observed and remains a critical clinical problem.

Thus, a need remains for identification of additional cancer biomarkers that are useful for detecting, diagnosing, monitoring, and for predicting responsiveness to cancer therapies and staging cancer.

### SUMMARY

The present application provides methods for detecting, diagnosing, monitoring, predicting responsiveness to cancer therapies and staging cancer. The present application also provides methods and compositions useful for suppression of GRP78 expression or function in breast cancer cells to overcome resistance of hormone receptor positive breast cancers. Methods for predicting whether a subject with breast cancer is at risk for developing resistance to hormonal therapy are provided.

The details of one or more embodiments are set forth in the accompanying drawings and the description below. Other features, objects, and advantages will be apparent from the description and drawings, and from the claims.

### DESCRIPTION OF DRAWINGS

FIGS. 1A to 1E show photomicrographs of Grp78 expression in prostate cancer. FIG. 1A shows a tumor from an untreated T₃N₀M₀ group showing >50% of tumor cells with moderately (2+) intense Grp78 cytoplasmic immunoreactivity; original magnification ×200. FIG. 1B shows a tumor from a treated T₃N₀M₀ group showing intense (3+) focal Grp78 cytoplasmic immunoreactivity; original magnification ×200. FIG. 1C shows the inset from FIG. 1B; subpopulations of prostate cancer cells demonstrate intense Grp78 cytoplasmic immunoreactivity (arrows). FIG. 1D shows a tumor from a castration resistant group showing high (3+) intensity cytoplasmic immunoreactivity; original magnification ×200. FIG. 1E shows immunostaining of Grp78 in LNCaP cells grown in FCS, LNCaP cells grown in androgen-depleted medium for six days (6DCSS), and C42B cells. AICS II assisted computer imaging analysis shows that C42B (84% tumor cells reactive) and LNCaP cells grown in CSS for 6 days (64.2% tumor cells reactive) showed higher Grp78 cytoplasmic immunoreactivity than cells grown in FCS (24.5% tumor cells reactive); percentages given as mean value of 5 representative areas+standard deviation. Negative control with no primary antibody is shown in the left panel; original magnification ×100; inset magnification ×200.

FIG. 2 shows grp78 expression in prostate cancer cells during brief and prolonged androgen starvation. Western blot analysis for Grp78 expression in LNCaP cells grown in fetal calf serum (FCS) and charcoal-stripped serum (CSS, two, four, and six days) and castration resistant C42B cells; β actin loading control shown in the lower panel; numbers represent the ratio of sample band intensity to β actin band intensity, using the lowest ratio (LNCaP FCS) as the reference point of 1.00.

FIG. 3 is a graph showing the probability of recurrence-free (clinical and/or PSA) status in 164 patients with stage T₃N₀M₀ prostate cancer, based on levels of Grp78 immunoreactivity. Untreated stage T₃N₀M₀ patients demonstrated greater probability of prostate cancer recurrence with higher Grp78 expression. Tick marks represent patients with no evidence of disease at last follow-up. The P value was obtained using the log-rank test.

FIG. 4 is a graph showing the probability of recurrence-free (clinical and/or PSA) status in 80 patients with stage T₃N₀M₀ prostate cancer stratified by median age, based on levels of Grp78 immunoreactivity. Untreated stage T₃N₀M₀ patients were stratified by age, where patients under the cohort median age of 67 years (n=80) demonstrated greater probability of prostate cancer recurrence with higher Grp78 expression. Tick marks represent patients with no evidence of disease at last follow-up. The P value was obtained using the log-rank test.

FIGS. 5A to 5D show selective association of endogenous BIK with GRP78. In FIG. 5A, 293T cells were either non-treated or treated with 50 μM etoposide (Etop) for 6 hours and were harvested 24 hours later. MCF-7/BUS cells were cultured either in regular DMEM or in estrogen-free DMEM for 48 hours. Western blots of total protein lysates from these cells were performed with antibodies against BIK and β-actin. In FIG. 5B, cell lysates prepared from control and Etop-treated 293T cells were immunoprecipitated with anti-BIK or normal IgG The immunoprecipitates were applied in parallel with input lysates to SDS-PAGE and Western blotted with antibodies against GRP78, GRP94, calnexin, calreticulin and BIK. FIG. 5C shows Coomassie blue staining of GST-GRP78, GST-BIK, and GST resolved by SDS-PAGE. In FIG. 5D, lysates of 293T cells were incubated with GST-GRP78, GST-BIK, or GST linked beads. The bound proteins were resolved by SDS-PAGE and probed for GRP78 or BIK by Western blotting.

FIGS. 6A to 6D show binding of GRP78 to ER-targeted BIK and suppression of its pro-apoptotic activity. In FIG. 6A, cell lysates prepared from 293T cells transfected with either empty vector pcDNA3 (−) or vector expressing Flag-BIK-b5™ (+) were immunoprecipitated with either anti-Flag antibody or normal IgG as a control. The immunoprecipitates were resolved by SDS-PAGE and Western blotted with anti-GRP78 and anti-Flag antibodies. For FIGS. 6B, 6C and 6D, 293T cells were transfected with empty vector(−), pcDNA3-Flag-BIK-b5TM, or pcDNA3-His-GRP78, alone or in combination as indicated. In FIG. 6B, the expression level of each protein was determined by Western blot. In FIG. 6C, the percent cell death in each transfection was assessed by trypan blue exclusion assay. In FIG. 6D, the percent of apoptotic cells was assessed by mitochondrial membrane potential staining. Columns in FIGS. 6C and 6D represent the mean from three experiments, four hundred cells for every group was assayed for each experiment. Standard error: * p<0.05, ** p<0.01.

FIGS. 7A to 7D show that overexpression of GRP78 rescues MCF-7/BUS cells from estrogen-starvation induced apoptosis. In FIG. 7A, cell lysates from MCF-7/BUS cells infected with adenoviral vector expressing GFP (Ad-GFP) or GRP78 (Ad-GRP78) cultured either in regular medium or in estrogen-free medium for 48 hours were subjected to SDS-PAGE and Western blots. The levels of GRP78, BIK, β-actin, the cleaved form of PARP (a signature of apoptosis) and the uncleaved form are indicated. FIG. 7B shows FACS analysis of the same samples in FIG. 7A using mouse anti-BAX and phycoerythrin-labeled anti-mouse antibodies. FIG. 7C shows mitochondrial membrane potential staining of MCF-7/BUS cells cultured either in regular medium or in estrogen-free medium after infection of adenovirus empty vector (Ad-Vector) or Ad-GRP78. FIG. 7D shows general morphology under light microscope of MCF-7/BUS cells at 0, 48 and 72 hours after estrogen starvation.

FIGS. 8A to 8D shows that knockdown of GRP78 sensitizes MCF-7/BUS cells to estrogen-starvation induced apoptosis. In FIG. 8A, cell lysates from MCF-7/BUS cells transfected with siGrp78 oligomers or control siRNA (siCtrl) for 24 hours and subsequently cultured in regular or estrogen-free medium (ES) for 24 hours were subjected to SDS-PAGE and Western blotting to probe for levels of GRP78, GRP94 and β-actin. In FIG. 8B, MCF-7/BUS cells were cultured either in regular or in estrogen-free (ES) medium for 24 hours after transfection of siGrp78 or siCtrl as indicated. The percent of apoptotic cells was assessed by mitochondrial membrane potential staining. The standard errors are indicated. In FIG. 8C, MCF-7/BUS cells were transfected with control siRNA, siGrp78, or siBik, alone or in combination as indicated for 24 hours and then cultured in ES medium for 24 hours. The total amount of siRNA in each condition was adjusted to be the same by addition of siCtrl. Cell lysates were collected and subjected to SDS-PAGE and probed for levels of GRP78, BIK and β-actin by Western blotting. In FIG. 8D, cell lysates from FIG. 8C were subjected to SDS-PAGE and Western blotted with anti-PARP antibody. The Western signal of full-length PARP and apoptosis-signature fragment were quantitated by Fluor-S™ MultiImager (Bio-Rad, Hercules, Calif.). The relative PARP cleavages are shown with the PARP cleavage in cells transfected with control siRNA set as 1. Columns in FIGS. 8B and 8D represent the mean from three or two experiments, respectively. Standard error: * p<0.05, ** p<0.01.

FIGS. 9A and 9B are photomicrographs of immunohistochemical staining of GRP78. Magnification, ×400. FIG. 9A shows negative staining for GRP78 in neoplastic cells of an infiltrating ductal carcinoma. Arrow, plasma cells stain intensely. FIG. 9B shows intense staining (3+) for GRP78 in neoplastic cells of an infiltrating ductal carcinoma.

FIGS. 10A to 10D show probability of remaining recurrence-free according to GRP78 expression in patients treated with Adriamycin-based adjuvant chemotherapy. FIG. 10A includes all 127 patients. FIG. 10B is a subset of 102 patients who did not receive taxanes (paclitaxel or docetaxel) as part of the Adriamycin-based regimen. FIG. 10C is a subset of 92 patients who underwent mastectomy. FIG. 10D is a subset of 74 patients who underwent mastectomy and did not receive taxanes as part of the regimen.

FIGS. 11A, 11B and 11C show specific detection of GRP78 by H129 antibody. FIG. 11A is a Western blot assay. The human neuroblastoma SK-N-SH cells were either grown under normal conditions (−) or treated with 0.5 μM thapsigargin for 16 hours. Fifty μg of total cell lysate prepared from these cells were subjected to Western blot analysis, using the anti-GRP78H1129 antibody (1:1000 dilution). The position of the single GRP78 protein band highly inducible by thapsigargin stress is indicated ( [symbol] ). In FIG. 11B, Chinese hamster ovary (CHO) cells expressing basal level of GRP78 and its derivative C.1 cells overexpressing GRP78 were embedded in paraffin after fixation in formalin. The sections prepared from these blocks were stained with the immunohistochemical technique using the H129 antibody (1:100 dilution). GRP78 level, as depicted by brown staining, was elevated in C.1 cells as compared to CHO cells (600×). In FIG. 11C, paraffin slides from breast cancer patients were stained with the H129 antibody with the immunohistochemical technique. Examples of plasma cell staining from two different patients are shown. The plasma cells showed uniform pattern of strong staining of GRP78 (600×).

FIGS. 12A and 12B are graphs of Q-PCR analysis of various cell lines under normal conditions (control, open bars) and following exposure to thapsigargin (TG, striped bars). FIG. 12A shows the levels of grp78 mRNA. FIG. 12B shows the levels of the mRNA splice variant of grp78 (78ISa).

### DETAILED DESCRIPTION

Resistance to castration therapies persists as the predominant challenge in the treatment of advanced prostate cancer. Androgen dependent prostate cancer is characterized by the ability of cancer cells to undergo apoptosis in response to hormone depletion. The transition to castration resistant prostate cancer (CR) requires the survival of tumor cells in such conditions, which may be attributed to a number of molecular mechanisms resulting in the evasion of apoptosis. One potential cellular survival mechanism in CR is through upregulation of stress response pathways, which confers protection to cells when they are subject to adverse conditions. The role of Grp78 in prostate cancer progression and the development of castration resistance (CR), where cancer cells continue to survive despite the stress of an androgen-starved environment is described. Classification of tumors was based on intensity of Grp78 cytoplasmic immunoreactivity and percent of immunoreactive tumor cells. The associations of Grp78 expression with prostate cancer recurrence (clinical and/or serum PSA) and survival were examined in the untreated stage T₃N₀M₀ group. The percentage of tumor cells expressing Grp78 was strongly associated with castration resistant status (p=0.005). Grp78 expression was also seen to be increased in the castration resistant LNCaP-derived cell line, C42B, and in LNCaP cells grown in androgen-deprived conditions, compared to LNCaP cells grown in androgen-rich media. Increased Grp78 expression was consistently associated with greater risk of prostate cancer recurrence and worse overall survival in patients who had not undergone prior hormonal manipulation. These data show that upregulation of Grp78 is associated with the development of CR and serves as an important prognostic indicator of recurrence in patients.

The recent development of hormonal therapy that blocks estrogen synthesis represents a major advance in the treatment of estrogen receptor positive breast cancer. However, cancer cells often acquire adaptations resulting in resistance. Estrogen-starvation induced apoptosis of breast cancer cells requires BIK, an apoptotic BH-3-only protein located primarily at the endoplasmic reticulum (ER). As described herein, it was discovered that BIK selectively forms complex with the glucose-regulated protein Grp78/BiP, a major ER chaperone with pro-survival properties induced in the tumor microenvironment. Grp78 overexpression decreases apoptosis of 293T cells induced by ER-targeted BIK. For estrogen-dependent MCF-7/BUS breast cancer cells, overexpression of Grp78 inhibits estrogen-starvation induced BAX activation, mitochondrial permeability transition, and consequent apoptosis. Further, knockdown of endogenous Grp78 by siRNA sensitizes MCF-7/BUS cells to estrogen-starvation induced apoptosis. This effect was substantially reduced when the expression of BIK was also reduced by siRNA. As shown in the Examples below, Grp78 confers resistance to estrogen-starvation induced apoptosis in human breast cancer cells. Thus Grp78 expression level in the tumor cells is a prognostic marker for responsiveness to hormonal therapy based on estrogen starvation, and combination therapy targeting Grp78 enhances efficacy and reduce resistance.

The discovery of predictive factors for chemoresistance is critical for improving adjuvant therapy for cancer patients. The 78-kDa glucose-regulated protein (Grp78), widely used as an indicator of the unfolded protein response (UPR), is induced in the tumor microenvironment. The present disclosure demonstrates that Grp78 confers resistance to chemotherapeutic agents such as, for example, topoisomerase inhibitors.

Thus, provided herein are methods of determining whether a subject with cancer is at risk for developing resistance to hormonal therapy. Provided herein are also methods of determining whether a subject with cancer is at risk for developing resistance to chemotherapy. Specifically, a method of determining whether a subject with cancer is at risk for developing resistance to hormonal therapy comprising selecting a subject at risk for developing resistance to hormonal therapy, obtaining a biological sample from the subject and determining the level of expression of Grp78 in the biological sample, wherein overexpression of Grp78 in the biological sample as compared to a control indicates that the subject is at risk for developing resistance to hormonal therapy. The subject can have breast cancer or prostate cancer. The prostate cancer can be androgen dependent. The breast cancer can be hormone receptor positive breast cancer.

The hormonal therapy of the provided methods includes, but is not limited to, anti-androgen agents such as, for example, finasteride and anti-estrogen agents such as for example, aromatase inhibitors or tamoxifen. Aromatase inhibitors include, but are not limited to, exemestane, aminoglutethimide, 4-androstene-3,6,17-trione, anastrozole and letrozole.

Methods of determining whether a subject with cancer is at risk for developing resistance to a chemotherapeutic agent are provided. Specifically, the methods include the steps of selecting a subject at risk for developing resistance to a chemotherapeutic agent, obtaining a biological sample from the subject and determining the level of expression of Grp78 in the biological sample, wherein overexpression of Grp78 in the biological sample as compared to a control indicates that the subject is at risk for developing resistance to the chemotherapeutic agent. Optionally, the subject has breast cancer and the chemotherapeutic agent is a topoisomerase inhibitor. Topoisomerase inhibitors include, but are not limited to, doxorubicin, irinotecan, topotecan, amsacrine, etoposide, etoposide phosphate and teniposide.

Grp78 can be detected by the methods described herein as well as nucleic-acid based detection methods such as RT-PCR and Northern blot and protein-based detection methods such as Western blot and Enzyme-Linked ImmunoSorbent Assay (ELISA), which are well known to those of skill in the art. Antibodies to Grp78 are commercially available, for example, from Santa Cruz Biotechnology (Santa Cruz, Calif.).

The application also provides a method of staging cancers in a subject. The stage of a cancer indicates how far a cancer has spread. Staging is important because treatment is often decided according to the stage of a cancer. The staging of a cancer has to do with the size of the tumor, and the degree to which it has penetrated. When the tumor is small and has not penetrated the mucosal layer, it is said to be stage I cancer. Stage II tumors are into the muscle wall, and stage III involves nearby lymph nodes. The stage IV cancer has spread (metastasized) to remote organs.

The method of staging cancer comprises identifying a subject having cancer and analyzing a sample of cells, tissues, or bodily fluid from such subject for Grp78. The measured Grp78 levels are then compared to levels of Grp78 in preferably the same cells, tissues, or bodily fluid type of a normal subject or control subject, wherein an increase in Grp78 levels in the subject versus a control subject is associated with a cancer which is progressing and a decrease in the levels of Grp78 is associated with a cancer which is regressing or in remission.

As an example, the methods described herein can be performed as follows. A biopsy or biological sample can be obtained from a subject. If the biological sample is a tissue sample it can be placed onto slides for Hematoxylin and Eosin (H&E) staining. An antibody against Grp78 (such as Grp78 H129, which binds amino acids 525 to 653 of human Grp78 and is from Santa Cruz Biotechnology (Santa Cruz, Calif.), or other antibody that binds Grp78) can be used in immunohistochemical staining of the slides for Grp78 levels. From the H&E staining, the location of the cancer on the slide can be determined. From the intensity of the Grp78 antibody staining the level of Grp78 in cancer cells can be determined. The level of Grp78 in cancer cells can be used to predict chemoresponsiveness to chemotherapy treatments and/or for staging the cancer.

Methods for treating castration resistant prostate cancer in a subject are provided. Such methods comprise the steps of selecting a subject at risk for developing resistance to hormonal therapy and contacting the castration resistant prostate cancer cells in the subject with one or more agents that inhibit expression or activity of GRP78 and a therapeutic agent. The therapeutic agent can be an anti-hormonal agent or a chemotherapeutic agent. Anti-hormonal agents include, for example, anti-androgen agents such as, for example, finasteride. Expression of Grp78 mRNA or Grp78 protein can be inhibited. For example, the grp78 gene or its promoter can be inactivated. Alternatively, the activity of Grp78 can be inhibited. Optionally, the agent that inhibits expression or activity of Grp78 is not a taxane.

Also provided are methods of treating hormone receptor positive breast cancer in a subject comprising selecting a subject at risk for developing resistance to hormonal therapy and contacting the hormone receptor positive breast cancer cells in the subject with one or more agents that inhibit expression or activity of Grp78 and a therapeutic agent. The therapeutic agent can be an anti-hormonal agent or a chemotherapeutic agent. Anti-hormonal agents include, for example, anti-estrogen agents such as, for example, aromatase inhibitors and tamoxifen. Aromatase inhibitors include, for example, exemestane, aminoglutethimide, 4-androstene-3,6,17-tone, anastrozole and letrozole. Expression of Grp78 mRNA or Grp78 protein can be inhibited. For example, the grp78 gene or its promoter can be inactivated. Alternatively, the activity of Grp78 can be inhibited. Optionally, the agent that inhibits expression or activity of Grp78 is not a taxane.

Provided are methods of treating breast cancer in a subject comprising selecting a subject at risk for developing resistance to a chemotherapeutic agent and contacting the breast cancer cells in the subject with one or more agents that inhibits expression or activity of Grp78 and a chemotherapeutic agent. Optionally, the agent that inhibits expression or activity of Grp78 is not a taxane.

The provided methods comprise administering an agent that reduces or inhibits expression or activity of Grp78. Reduction or inhibition of Grp78 can comprising inhibiting or reducing expression of Grp78 mRNA or Grp78 protein, such as by administering antisense molecules, triple helix molecules, ribozymes and/or siRNA. grp78 gene expression can also be reduced by inactivating the grp78 gene or its promoter. The nucleic acids, ribozymes, siRNAs and triple helix molecules for use in the provided methods may be prepared by any method known in the art for synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the nucleic acid molecule. Such DNA sequences may be incorporated into a wide variety of vectors, which incorporate suitable RNA polymerase promoters. Antisense cDNA constructs that synthesize antisense RNA constitutively or inducibly, depending on the promoter used, can be introduced stably into cell lines.

In addition, reduction or inhibition of Grp78 includes inhibiting the activity of the Grp78 protein, referred to herein as Grp78 antagonists. Drugs which target Grp78 have been developed (Ermakova et al., Cancer Res. 66:9260-9 (2006); Davidson et al., Cancer Res. 65:4663-72 (2005); Zhou et al., J. Natl. Cancer Inst. 90:381-88 (1998); Arap et al., Cancer Cell 6:275-84 (2004); Park et al., J. Natl. Cancer Inst. 96:1300-10). Grp78 antagonists also include antibodies, soluble domains of Grp78 and polypeptides that interact with Grp78 to prevent Grp78 activity. The nucleic acid and amino acid sequence of Grp78 is known in the art. Therefore, variants and fragments of Grp78 that act as Grp78 antagonists can be prepared by any method known to those of skill in the art using routine molecular biology techniques. Numerous agents for modulating expression/activity of intracellular proteins such as GRP in a cell are known. Any of these suitable for the particular system being used may be employed. Typical agents for inhibiting or reducing (e.g., antagonistic) activity of GRPs include mutant/variant GRP polypeptides or fragments and small organic or inorganic molecules.

Thus, the present application provides methods and compositions useful for targeted suppression of GRP expression or function in breast cancer cells to overcome resistance of hormone receptor positive breast cancers to hormonal therapy.

Inhibitors of Grp78 include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more. Inhibitory peptides include chimeric peptides with Grp78 binding motifs fused to pro-apoptotic sequences (Arap et al., Cancer Cell 6:275-84 (2004), which is incorporated by reference herein in its entirety), Inhibitory proteins also include Kringle 5 (K5), melanoma differentiation-associated gene-7/interleukin-24 (MDA7/IL-24) and activated form of α-2 macroglobulin (Davidson et al., Cancer Res. 65:4663-72 (2005); Dent et al., J. Cell Biochem. 95:712-9 (2005); Misra et al., J. Biol. Chem. 281:3694-707 (2006), which are incorporated by reference herein in their entireties).

Inhibitory peptides include dominant negative mutants of a Grp78. Dominant negative mutations (also called antimorphic mutations) have an altered phenotype that acts antagonistically to the wild-type or normal protein. Thus, dominant negative mutants of Grp78 act to inhibit the normal Grp78 protein. Such mutants can be generated, for example, by site directed mutagenesis or random mutagenesis. Proteins with a dominant negative phenotype can be screened for using methods known to those of skill in the art, for example, by phage display.

Nucleic acids that encode the aforementioned peptide sequences are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants. Such peptides or proteins are selected based on their ability to reduce or inhibit expression or activity of Grp78.

Inhibitors of a Grp78 also include, but are not limited to, genistein, (−)-epigallocatechin gallate (EGCG), salicyclic acid from plants, bacterial AB₅ subtilase cytoxin, versipelostatin (Ermakova et al., Cancer Res. 66:9260-9 (2006); Zhou and Lee, J. natl. Cancer Inst. 90:381-8 (1998); Deng et al., FASEB J 15:2463-70 (2001); Montecucco and Molinari, Nature 443:511-2 (2006); Park et al., J. Natl. Cancer Inst. 96:1300-10 (2004), which are incorporated herein in their entireties). Inhibitors of GRP78 also include taxanes, such as, for example, paclitaxel and docetaxel in combination with doxirubicin.

Also provided herein are functional nucleic acids that inhibit expression of Grp78. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of Grp78.

Functional nucleic acids are nucleic acid molecules that have a specific unction, such as binding a target molecule or catalyzing a specific reaction. Functional nucleic acid molecules can interact with any macromolecule, such as DNA, RNA, polypeptides, or carbohydrate chains. Thus, functional nucleic acids can interact with the mRNA, genomic DNA, or polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855, 5,877,022, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules can be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

Proteins that inhibit Grp78 include antibodies with antagonistic or inhibitory properties. Antibodies to Grp78 are commercially available, for example, from Santa Cruz Biotechnology (Santa Cruz, Calif.). In addition to intact immunoglobulin molecules, fragments, chimeras, or polymers of immunoglobulin molecules are also useful in the methods taught herein, as long as they are chosen for their ability to inhibit Grp78. The antibodies can be tested for their desired activity using in vitro assays, or by analogous methods, after which their in vivo therapeutic or prophylactic activities are tested according to known clinical testing methods.

The term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

Digestion of antibodies to produce fragments thereof, e.g., Fab fragments, can be accomplished using routine techniques known in the art. For instance, digestion can be performed using papain. Examples of papain digestion are described in WO 94/29348 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggerman et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen binding portion of an antibody. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucheriapati et al.), U.S. Pat. No. 6,130,364 (Jakobovits et al.), and U.S. Pat. No. 6,180,377 (Morgan et al.).

The compositions and agents that reduce or inhibit Grp78 are optionally administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The agent or compositions may be administered orally, parenterally (e.g., intravenously), by intramuscular injection, by intraperitoneal injection, transdermally, extracorporeally, topically or the like, including intranasal administration or administration by inhalant. The dosage of the agent or composition required will vary from subject to subject, depending on the species, age, weight and general condition of the subject, the severity of the airway disorder being treated, the particular active agent used, its mode of administration and the like. Thus, it is not possible to specify an exact amount for every composition. However, an appropriate amount can be determined by one of ordinary skill in the art using only routine experimentation given the teachings herein.

Suitable carriers and their formulations are described in Remington: The Science and Practice of Pharmacy (21st ed.) eds. A. R. Gennaro et al., University of the Sciences in Philadelphia 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-cancer compound, anti-inflamatory agents, anti-viral agents, anti-retroviral agents, anti-opportunistic agents, antibiotics, immunosuppressive agents, immunoglobulins, and antimalarial agents.

An anti-cancer compound or chemotherapeutic agent is a compound or composition effective in inhibiting or arresting the growth of an abnormally growing cell. Thus, such an agent may be used therapeutically to treat cancer as well as other diseases marked by abnormal cell growth. A pharmaceutically effective amount of an anti-cancer compound is an amount administered to an individual sufficient to cause inhibition or arrest of the growth of an abnormally growing cell. Illustrative examples of anti-cancer compounds include: bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.

Any of the aforementioned treatments can be used in any combination with the compositions described herein. Thus, for example, the compositions can be administered in combination with a chemotherapeutic agent and radiation. Other combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

As used herein, references to decreasing, reducing, or inhibiting include a change of 10, 20, 30, 40, 50, 60, 70, 80, 90 percent or greater as compared to a control level. Such terms can include but do not necessarily include complete elimination.

As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of the severity of one or more symptoms of the disease or disorder.

There are a variety of sequences related to, for example, Grp78 that are disclosed on Genbank, at www.pubmed.gov, and these sequences and others are herein incorporated by reference in their entireties as well as for individual subsequences contained therein.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a biomarker is disclosed and discussed and a number of modifications that can be made to a number of molecules including the biomarker are discussed, each and every combination and permutation of the biomarker and the modifications that are possible are specifically contemplated unless specifically indicated to the contrary. Thus, if a class of molecules A, B, and C are disclosed as well as a class of molecules D, E, and F and an example of a combination molecule, A-D, is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Throughout this application, various publications are referenced. The disclosures of these publications in their entireties are hereby incorporated by reference into this application in order to more fully describe the state of the art to which this invention pertains.

A number of embodiments of the invention have been described. Nevertheless, it will be understood that various modifications may be made. Furthermore, when one characteristic or step is described it can be combined with any other characteristic or step herein even if the combination is not explicitly stated. Accordingly, other embodiments are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

### Example 1

### Expression of Grp78 is Associated with Development of Castration-Resistant (CR) Prostate Cancer

Materials and Methods.

Patient population. The recruitment and studies of patients described here have been approved by local institutional review boards (HS #-006044). This study included tumor samples from 219 patients with prostate cancer, comprised of three distinct cohorts of patients. One hundred ninety-one patients were classified as pathologic stage T₃N₀M₀ disease, and specimens were obtained through radical retropubic prostatectomy with bilateral pelvic lymph node dissection at the University of Southern California/Norris Comprehensive Cancer Center between 1982 and 1996. These patients were further subdivided according to treatment status. Treatment consisted of neoadjuvant androgen ablation therapy with 1 mg diethylstilbestrol two or four times per day for 3 days to 20 weeks before radical prostatectomy. The stage T₃N₀M₀ untreated group included 164 patients who were not exposed to preoperative androgen ablation therapy. The group of 27 men comprising the stage T3N0M0 treated group had received neoadjuvant androgen ablation therapy, and these patients were considered responsive to androgen (Frader, Urol. Clin. North Am. 23:575-85 (1996)). Tumor samples were obtained from 28 patients with castration resistance who underwent hormone ablation via orchidectomy and systemic hormone therapy but continued to show increasing prostate-specific antigen (PSA). Between 1990 and 1992, these men underwent transurethral resection to relieve urinary obstruction at Ruhr University, Bochum, Germany. All tumor grading was in concordance with the Gleason system.

Patient follow-up. Evaluations of the T₃N₀M₀ patients were done at 1, 2, and 6 months postoperatively, at 6-month intervals for 5 years following surgery, then yearly. Biopsy was used to assess clinical recurrence of prostate cancer, and metastatic disease was determined according to bone scan or alternate clinical findings. PSA recurrence was designated to patients showing serum PSA levels ≧0.4 ng/mL on two consecutive tests. Median follow-up in the untreated group of 164 patients was 12.7 years with a range of 1.6 to 20 years. Median age was 67 years, ranging from 47 to 81 years.

Immunohistochemistry. Formalin-fixed 5-μm sections were taken from paraffin-embedded prostate cancer specimens and cell lines and mounted on poly-L-lysine-coated slides. The slides were deparaffinized in xylene, washed with 100% ethanol, followed by rehydration in 95% ethanol; 3% hydrogen peroxide in absolute methanol was used to quench endogenous peroxidase. Antigen retrieval was done using citrate buffer (pH 6) and microwaving for 30 minutes followed by cooling at room temperature for 20 minutes. The slides were then blocked with normal horse serum for 20 minutes and incubated for 1 hour with anti-Grp78 rabbit polyclonal antibody (Santa Cruz Biotechnology, SantaCruz, Calif.) at a 1:100 dilution in PBS. Incubation with biotinylated horse anti-rabbit secondary antibody at a 1:200 dilution was followed by avidin-biotin conjugate (Vector Laboratories, Inc., Burlingame, Calif.). Chromogen of 0.03% diaminobenzidine was then applied, with hematoxylin counterstaining. Negative controls consisting of diluent with no antibody and positive prostate cancer controls with heterogeneous immunoreactivity were used in all experiments.

Cultured prostate cancer cells (LNCaP and C42B) were harvested, cytospun on poly-L-lysine-coated slides at 250,000 per slide, and formalin fixed. Antigen retrieval was done using citrate buffer (pH 6) and microwaving for 5 minutes followed by cooling at room temperature for 15 minutes. Subsequent steps in immunohistochemistry protocol follow as described above.

Immunoreactivity assessment of clinical samples. All slides were interpreted by two pathologists, who were blinded to all outcome data. Tumor scores were categorized based on two criteria: (a) percentage of tumor cells showing cytoplasmic immunoreactivity and (b) intensity of cytoplasmic immunostaining. For assessment according to percentage of cytoplasmic reactivity, tumors were classified as showing low Grp78 expression (≦50%) or high Grp78 expression (≧50%). For intensity of cytoplasmic immunoreactivity, tumors were classified as having low Grp78 expression (≧1), moderate Grp78 expression (≧2), or high Grp78 expression (≧3). Grp78 status was assigned as negative to cases with <10% Grp78 immunoreactivity or weak (≧1) staining. All other cases were assigned positive Grp78 status. Upon identification of focal areas where Grp78 expression levels were markedly intense, tumors were further categorized by percentage of cells showing intense (≧3) Grp78 immunoreactivity (<5%, low Grp78; ≧5%, high Grp78). Due to the heterogeneity of Grp78 immunoreactivity, scoring corresponds with an overall evaluation of the entire tissue section. Lymphocytes, which are highly immunoreactive with anti-Grp78, were used as internal positive controls.

Cell culture. Androgen-responsive LNCaP and androgen-resistant LNCaP-derived C42B cells were grown in RPMI 1640 (Invitrogen, Carlsbad, Calif.) with 50 units/mL penicillin, 50 units/mL streptomycin, and 10% FCS (Mediatech, Inc., Herndon, Va.). For preparation of androgen-depleted medium, FCS and RPMI 1640 were replaced by 10% dextran/charcoal-stripped serum (Omega Scientific, Inc., Tarzana, Calif.) and phenol-free RPMI 1640 (Invitrogen, Carlsbad, Calif.), as previously described (Craft et al., Nat. Med. 5:280-5 (1999)). All cell lines were maintained in a humidified incubator at 5% CO₂ and 37° C.

Automated cellular imaging. Immunostaining and evaluation of immunostained cell lines were carried out in triplicate, where immunoreactivity was assessed using ACIS II (Clarient, Inc., Aliso Viejo, Calif.). The ACIS II system consists of a computer-assisted bright-field microscope (×4, ×10, ×20, ×40, and ×60 objectives) coupled to a SONY 3-chip CCD camera. This fully automated system creates a reconstructed image of an immunohistochemistry stained slide and uses wavelength-specific technology to detect color differences between objects. Immunostained slides of cytospun cell lines were scanned at ×4 magnification followed by image capture, transformation to pixels, and quantification by hue (color), saturation (color purity), and luminosity (brightness). Five regions of interest at ×4 magnification were manually selected for each sample slide, and brown color (3,3¶-diaminobenzidine chromogen) was assessed by ACIS software, which counts pixels based on 256 levels of color intensity. Representative areas were analyzed for intensity and percentage of cells positive for brown color.

Western blot analysis. For Western blot analysis, cell lysates from LNCaP and C42B cells were prepared by lysing in 1 mL ice-cold radio immunoprecipitation assay buffer. Equal amounts of total protein from each sample were subjected to SDS-PAGE in a 7.5% Tris-HCl gel (Bio-Rad Laboratories, Hercules, Calif.). Following electrophoresis, the proteins were transferred to a pure nitrocellulose membrane (Bio-Rad Laboratories, Hercules, Calif.). The membrane was then incubated in Odyssey® blocking buffer (Li-Cor Biosciences, Lincoln, Nebr.) followed by overnight incubation with primary rabbit polyclonal anti-Grp78 antibody (1:500 dilution; Santa Cruz Biotechnology, Santa Cruz, Calif.). Signal detection was done using Alexa Fluor® 680 goat anti-rabbit antibody (Molecular Probes, Eugene, Oreg.) and subsequent scanning of the membrane by the Odyssey® Infrared Imager (model 9120, Li-Cor Biosciences, Lincoln, Nebr.). All bands from Western analysis were quantified for protein expression with Odyssey® Infrared Imaging Software (Li-Cor Biosciences, Lincoln, Nebr.) to assess integrated intensity (pixel volume) as a measure of absorbance. Band density was represented as the ratio of average band intensity (I) of each sample to the average band intensity of the corresponding h-actin control band.

Statistical analysis. χ² and Fisher's exact tests were used to compare differences in Grp78 expression among all groups; if Ps for the overall test were significant at the 0.05 level, then these were used to analyze pairwise comparisons. Kaplan-Meier plots and the log-rank test were used to analyze the association of Grp78 expression with time to clinical and/or serum PSA recurrence and survival in the untreated stage T₃N₀M₀) group; the stratified log-rank test was used for multivariable analyses. Results were considered significant at P<0.05 for two-sided analyses.

Results.

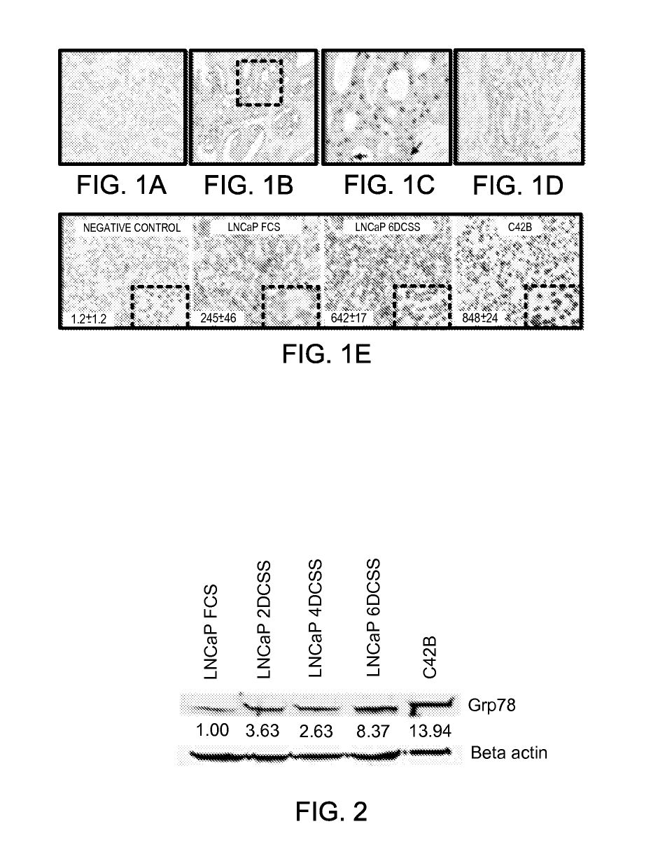

Grp78 expression in localized prostate cancer. Immunohisistochemistry was employed to evaluate Grp78 protein levels in tumors from 164 stage T₃N₀M₀ untreated and 27 stage T₃N₀M₀ treated prostate cancer patients. In the untreated group, 120 of 164 cases (73%) showed high Grp78 expression by percentage of cytoplasmic immunoreactivity (>50% stained tumor cells), as shown in FIGS. 1A to 1E and Table 1.

TABLE 1
GRP78 expression (immunoreactivity) in untreated T₃N₀M₀, treated
T₃N₀M₀, and castration-resistant prostate cancer.

| | Percentage Tumor | | | Intensity (%) | | |
|---|---|---|---|---|---|---|
| | Cells Reactive (%) | | | | Moderate/ | |
| | Low | High | | | High | |
| Tumor | (≦50%) | (>50%) | P* | Low (≧1) | (2 to >3) | P* |
| Untreated¹ | 44 (27) | 120 (73) | 0.002 | 73 (45) | 91 (55) | 0.033 |
| Treated² | 9 (33) | 18 (67) | <0.001 | 13 (48) | 14 (53) | 0.053 |
| CR³ | 0 (0) | 28 (100) | | 6 (21) | 22 (79) | |

P* values represent significant difference from castration-resistant group.
¹Tumors from stage T₃N₀M₀ prostate cancer patients who have not undergone preoperative androgen ablation therapy.
²Tumors from stage T₃N₀M₀ prostate cancer patients who have undergone preoperative androgen ablation therapy.
³Tumors from patients with castration-resistant prostate cancer.

Of the 27 cases in the treated group, however, 18 (67%) cases showed high Grp78 percent immunoreactivity (FIGS. 1A to 1E; Table 1). According to intensity of Grp78 immunoreactivity, 91 of 164 (55%) untreated cases showed moderate to high expression of Grp78 (FIGS. 1A to 1E; Table 1). In the treated group, 14 of 27 (52%) tumors showed moderate to high Grp78 expression (FIGS. 1A to 1E; Table 1). For percent immunoreactivity and intensity, the differences between the untreated and treated groups did not reach statistical significance (P=0.484 and P=0.913).

Focal areas of cells showing intense Grp78 immunoreactivity (≧5%; ≧3 intensity) were identified in both the untreated and treated stage T₃N₀M₀ cases (FIGS. 1B and 1C). In the untreated cases, 36% (59 of 164) versus 44% (12 of 27) of treated cases were classified as positive for this intense focal immunoreactivity.

Grp78 expression in castration-resistant prostate cancer. Of the 28 castration-resistant tumors immunostained for Grp78, 28 (100%) showed high Grp78 expression by percent cytoplasmic immunoreactivity (FIGS. 1A to 1E; Table 1). Compared with the untreated and treated stage T₃N₀M₀ cases, Grp78 expression according to percentage of immunoreactive tumor cells was significantly increased in castration resistance (P=0.005). This elevation in Grp78 expression remained significant even when comparing castration-resistant cases to the untreated and treated groups separately (P=0.002 and P<0.001). When Grp78 expression was examined in castration-resistant tumors by intensity of cytoplasmic immunoreactivity, 22 of 28 (79%) cases showed moderate to high expression (FIGS. 1A to 1E; Table 1). Compared with the stage T₃N₀M₀ cases, the number of tumors showing moderate to high intensity Grp78 expression in the castration-resistant group was significantly greater than both the untreated group (P=0.033) and the treated group (P=0.053). Furthermore, when Grp78 expression was examined as a combined measure of percentage of overall immunoreactive tumor cells and intensity (Grp78 status), Grp78 expression remained significantly elevated in the castration-resistant group when compared with both the untreated group (P=0.018) and the treated group (P=0.037).

In vitro expression of Grp78 corroborates clinical observations. Cell line models consisted of LNCaP-deprived castration-resistant C42B cells and androgen-dependent LNCaP cells grown in medium with FCS or in androgen-deprived conditions where FCS was replaced with charcoal-stripped serum. It was observed that C42B cells and LNCaP cells maintained in medium with charcoal-stripped serum (androgen depleted) for 6 days showed prominent cytoplasmic Grp78 immunoreactivity compared with LNCaP cells grown with FCS, which showed faint cytoplasmic Grp78 immunostaining (FIG. 1E). Quantitation of Grp78 cytoplasmic immunoreactivity by AICS II computer imaging of five representative areas on each sample slide showed that C42B cells had a mean of 84.0% immunoreactive tumor cells; LNCaP cells grown in charcoal-stripped serum for 6 days showed a mean of 64.2% reactive tumor cells; and LNCaP cells grown in FCS were found to have an average of 24.5% tumor cells showing cytoplasmic reactivity to Grp78 antibody. The ACIS II system reported a mean of 1.2% reactive tumor cells for the negative control LNCaP FCS cells excluding primary antibody. Intensity of each sample analyzed by ACIS II was also found to be greater in C42B and 6-day hormone-starved LNCaP cells than in LNCaP cells grown in FCS. As shown in FIG. 2 and Table 2, these results were corroborated by Western blot analysis of cell lysates prepared from LNCaP cells grown with FCS; LNCaP cells grown with charcoal-stripped serum for 2, 4, and 6 days; and C42B cells.

TABLE 2
Quantification of protein expression
from Western blot analysis of GRP78.

| | Average | Average | | |
|---|---|---|---|---|
| | Intensity of | Intensity of β- | | |
| Cell Line | Grp78 band | actin band | Ratio | Standardized |
| Sample | (I)¹ | (I_{β})¹ | I/I_{β}¹ | Ratio I/I_{β}^{1,2} |
| LNCaP FCS³ | 23.39 | 142.58 | 0.16 | 1.00 |
| LNCaP 2DCSS⁴ | 52.84 | 91.36 | 0.58 | 3.63 |
| LNCaP 4DCSS⁴ | 37.59 | 90.27 | 0.42 | 2.63 |
| LNCaP 6DCSS⁴ | 139.37 | 103.78 | 1.34 | 8.37 |
| C42B⁵ | 169.61 | 75.90 | 2.23 | 13.94 |

¹All measurements taken as average band intensity with data units of absorbance.
²Standardized ratio calculated using lowest ratio I/I_{β} as reference point of 1.00.
³Androgen-dependent LNCaP cells grown in medium supplemented with 10% fetal calf serum (FCS).
⁴LNCaP cells grown for 2, 4, or 6 days in androgen-depleted conditions of medium supplemented with 10% charcoal-stripped serum (CSS).
⁵LNCaP-derived castration-resistant C42B cell line.

Comparison of Grp78 protein levels, expressed as band intensity ratios, showed that Grp78 expression in LNCaP cells was lowest in cells grown with FCS (1.00 standardized ratio), increased upon androgen starvation for 2 and 4 days (3.63 and 2.63 ratios), even further increased upon 6 days of hormone depletion (8.37 ratio), and was highest in castration-resistant C42B cells (13.94 ratio).

Association of Grp78 expression with prostate cancer recurrence and survival. To evaluate Grp78 as a potential marker of prostate cancer progression, the association of Grp78 expression with cancer recurrence risk and overall survival in untreated stage T₃N₀M₀ patients was examined. Treated cases were excluded due to potential alterations in Grp78 expression as a result of exposure to hormone ablation. Untreated cases were stratified by age, PSA level, and Gleason grade. The associations between Grp78 expression and prostate cancer recurrence and survival in untreated stage T₃N₀M₀ patients (n=164, Table 3) were examined.

TABLE 3
Grp78 expression and recurrence-free or overall survival
of patients with untreated stage T₃N₀M₀ tumors.

| | Grp78 | Recurrence-free | |
|---|---|---|---|
| | expression¹ | survival², relative | Overall survival, |
| Variable | (n) | risk³ | relative risk³ |
| Total (n = 164) | | | |
| Grp78 < 5% | 105 | 1.00 | 1.00 |
| Grp78 ≧ 5% | 59 | 1.43 | 1.42 |
| Adjusted for age (y) | | | |
| ≦67 | 29/51 | 2.22 | 1.28 |
| >67 | 30/54 | 0.87 | 1.55 |
| Stratified | | 1.47 | 1.42 |
| Adjusted for PSA⁴ | | | |
| (ng/dL) | | | |
| <4 | 4/9 | 5.87 | 0.61 |
| 4-10 | 10/24 | 1.45 | 1.11 |
| 10-20 | 15/18 | 1.44 | 1.88 |
| >20 | 13/17 | 1.11 | 0.85 |
| Stratified | | 1.33 | 1.31 |
| Adjusted for | | | |
| Gleason score | | | |
| 2-4 | 1/7 | NA | 15.09 |
| 5-6 | 20/43 | 1.06 | 1.40 |
| 7-10 | 38/55 | 1.30 | 1.17 |
| Stratified | 38/55 | 1.24 | 1.30 |

Abbreviation: NA, not available.
¹Percentage (<5% or ≧5%) of tumor ceils with ≧3 intense immunoreactivity represents Grp78 expression.
²Recurrence includes clinical and/or PSA recurrence.
³Hazards ratios were calculated as a measure of relative risk.
⁴Number of patients with ≧5% tumor cells with ≧3 + Grp78/number of patients with <5% tumor cells with ≦3 Grp78.
⁵PSA values were not available for 54 patients who were excluded from PSA-stratified analyses.

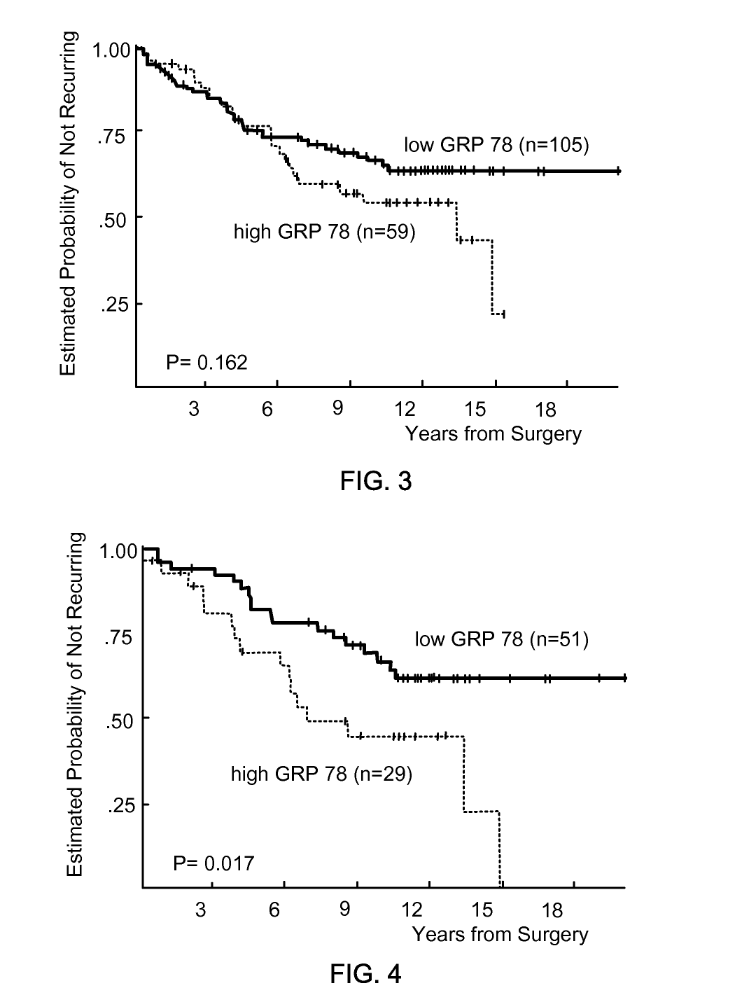

At median follow-up of 12 years in the stage T₃N₀M₀ untreated cohort, the probability of remaining recurrence free in cases expressing low Grp78 (<5% cells with intense immunoreactivity to Grp78) was 64% versus 54% in those expressing high (≧5% cells with intense immunoreactivity) levels of Grp78 (FIG. 3). Stratification of Grp78 expression in the stage T₃N₀M₀ untreated cohort, by the standard clinical variables (multivariable analyses adjusting for age, PSA measurements, and Gleason score), consistently showed that the risk of recurring or dying was greater for patients with tumors that expressed high levels of Grp78 (≧5% of tumor cells with ≧3 intensity) compared with patients with tumors that expressed low levels of Grp78, even after adjusting for these known predictors of outcome. Thus, as shown in Table 3, the relative risks, which compare patients with high Grp78 expression in tumors with those with low Grp78 expression in tumors, were not changed substantially after stratification. Although these trends did not achieve statistical significance at the 0.05 level, they are consistent across strata, for both recurrence and survival. Grp78 expression proved to be significant, however, among particular subsets of patients. It was observed that in the untreated stage T₃N₀M₀ patients who were below the median age of 67 (n=80) at diagnosis, increased Grp78 immunoreactivity (≧5% cells expressing high levels of Grp78) was significantly associated with increased risk of clinical and/or PSA recurrence (FIG. 4; Table 3). In these cases, the probability of remaining recurrence free in cases expressing low Grp78 was 61% versus 45% in those expressing high levels of Grp78, at follow-up year 12. The median recurrence-free interval for patients with low versus high Grp78 expression was 14.5 versus 8.7 years.

### Example 2

### GRP78 Protects Human Breast Cancer Cells Against Estrogen Starvation-Induced Apoptosis

Materials and Methods.

Cell lines and culture conditions. The estrogen-dependent cell line MCF-7/BUS was provided by A. M. Soto (Tufts University, Medford, Mass.) and has been described (Soto et al., Environ. Health Perspect. 103:113-22 (1995)). The human embryonic kidney 293T cells and MCF-7/BUS cells were maintained in DMEM supplemented with 10% fetal bovine serum. Estrogen starvation of MCF-7/BUS cells was done as described (Hur et al., PNAS 101:2351-6 (2004)). Briefly, the cells were washed thrice with phenol red-free DMEM and incubated in washing medium at 37° C. for 60 minutes. The MCF-7/BUS cells were then cultured in phenol red-free DMEM supplemented with 5% charcoal/dextran-stripped fetal bovine serum for 24 to 72 hours as indicated. For etoposide treatment, the cells were incubated with 50 μmol/L etoposide for 6 hours and cultured for another 24 hours before harvest.

Expression vectors. The plasmids pcDNA3-Flag-BIK-b5TM and pcDNA3-Flag-BIK and their construction have been described (Germain et al., J. Bio. Chem. 277:18053-60 (2002)). In pcDNA3-Flag-BIK-b5TM, the COOH-terminal transmembrane domain of BIK was replaced by the transmembrane domain of cytochrome b5, which targets the protein to the ER. The construction of pcDNA3-His-Grp78 has been described (Zeng et al., EMBO J. 23:950-8 (2004)).

Transient transfections and adenovirus infections. 293T cells were grown to 60% to 80% confluence. Two micrograms of pcDNA3-Flag-BIKb5TM plasmid were cotransfected with 2 μg of His-Grp78 or empty vector by using Polyfect (Qiagen, Valencia, Calif.) as described (Lee, Methods 35:373-81 (2005)). The green fluorescent protein (GFP) gene driven by cytomegalovirus promoter was added to monitor for transfection efficiency. Empty vector was added to adjust the total amount of plasmids to be the same. Forty-eight hours later, the transfected cells were subjected to cell death assays, Western blot, or coimmunoprecipitation.

For construction of the adenovirus expression vectors, either GFP or a His-tagged full-length hamster Grp78 cDNA was subcloned into an adenoviral vector and its expression was driven by the cytomegalovirus promoter. The sequence in the final construct was confirmed by DNA sequencing. MCF-7/BUS cells were infected at 100 plaque-forming units/cell with adenovirus vectors expressing GFP or Grp78. For mitochondrial membrane potential staining, because GFP interferes with the green fluorescence of this assay, the adenovirus empty vector was used as the negative control. After 24 hours, the infected cells were subjected to estrogen starvation for 48 hours. Each transfection or infection was done in duplicate and was repeated two to three times.

Western blots and quantitation. The Western blots were done as described (Lee, Methods 35:373-81 (2005)). The primary antibodies were goat anti-BIK (N-19, Santa Cruz Biotechnology, Santa Cruz, Calif.), rat anti-Grp78 (76-E6, Santa Cruz Biotechnology), rat anti-GRP94, rabbit anti-calnexin, rabbit anti-calreticulin (Stressgen, Ann Arbor, Mich.), mouse anti-Flag M2, mouse anti-poly(ADP-ribose) polymerase (PARP; F-2, Santa Cruz Biotechnology, Santa Cruz, Calif.), and mouse anti-h-actin (Sigma-Aldrich, St. Louis, Mo.). Anti-h-actin was diluted at 1:2,000; anti-BIK at 1:500; and other antibodies at 1:1,000. Respective horseradish peroxidase-conjugated secondary antibodies (Santa Cruz Biotechnology, Santa Cruz, Calif.) at 1:1,000 dilution were used. The Western blots were quantitated by Fluor-S® MultiImager (Bio-Rad, Hercules, Calif.) according to the manufacturer's instructions. All quantitations were normalized against h-actin.

Communoprecipitation assays. The coimmunoprecipitation assays were done as described (Reddy et al., J. Biol. Chem. 278:20915-24 (2003)). Briefly, 500 μg of total protein extract from each sample were pretreated with protein G-Sepharose beads (Upstate, Chicago, Ill.), followed by incubation with 5 Ag of goat anti-BIK antibody (N-19, Santa Cruz Biotechnology, Santa Cruz, Calif.) or mouse anti-Flag M2 antibody (Sigma-Aldrich, St. Louis, Mo.). For negative controls, the respective goat or mouse immunoglobulin G (IgG; Santa Cruz Biotechnology, Santa Cruz, Calif.) was used.

Glutathione S-transferase pull-down assays. Glutathione S-transferase (GST)-Grp78 and GST-BIK were constructed by subcloning full-length hamster Grp78 cDNA and human BIK into the BamH1/XhoI and BamH1/Sal1 sites of pGEX 4T1, respectively (Pharmacia Biotech, Piscataway, N.J.). Conditions for the GST pull-down assays have been described (Wu and Lee, Nucleic Acids Res. 26:4837-45 (1998)) with the following modifications. Five micrograms of GST-BIK, GST-Grp78, and GST bound to glutathione-Sepharose beads (Sigma-Aldrich, St. Louis, Mo.) were incubated with 500 μg of total protein extract on a rotating shaker at 4° C. for 16 hours. The beads were collected by centrifugation at 2,000 rpm for 5 min and washed thrice with extraction buffer. The bound proteins were eluted in SDS-PAGE sample loading buffer and subjected to SDS-PAGE and Western blotting.

Cell death and apoptotic assays. The cell death trypan blue exclusion assay was done as described (Dong et al., Cancer Res. 65:5785-91 (2005)). For mitochondrial membrane potential staining, the Mitochondrial Permeability Transition Detection Kit (Immunochemistry, Bloomington, Minn.) was used following the manufacturer's protocol. The cell cultures were then washed with PBS and examined under a fluorescence microscope. Each assay was done in triplicate.

Flow cytometric analysis of BAX-associated immunofluorescence. On initiation of apoptosis, BAX undergoes conformational change that exposes an otherwise inaccessible NH2-terminal epitope. A mouse monoclonal antibody against amino acids 12 to 24 (clone 6A7, PharMingen, San Diego, Calif.) was used to detect the BAX with proapoptotic conformational change. MCF-7/BUS cells were harvested and fixed in 0.25% paraformaldehyde in PBS for 5 minutes. BAX staining and fluorescence-activated cell sorting (FACS) analysis of BAX activation were done as described (Mandic et al., Mol. Cell. Biol. 21:3684-91 (2001)).

Small interfering RNA. The siRNA against Grp78 is 5′-gagcgcauugauacuagadTdT-3′ (SEQ ID NO:1) as described (Tsutsumi et al., Oncogene 25:1018-29 (2006)). The siRNA against Bik is 5′-aagaccccucuccagagacau-3′ (SEQ ID NO:2) (Hur et al., PNAS 101:2351-6 (2004)). The control siRNA is Silencer Negative Control #3 siRNA (Ambion, Foster City, Calif.) composed of a 19-bp scrambled sequence without significant homology to any known gene sequences from mouse, rat, or human. MCF-7/BUS cells were grown to 50% confluence and transfected with control siRNA or siRNA against Grp78 or Bik using Lipofectamine™ 2000 transfection reagent (Invitrogen, Carlsbad, Calif.) according to the manufacturer's instructions. The experiments were repeated two to three times.

Results.

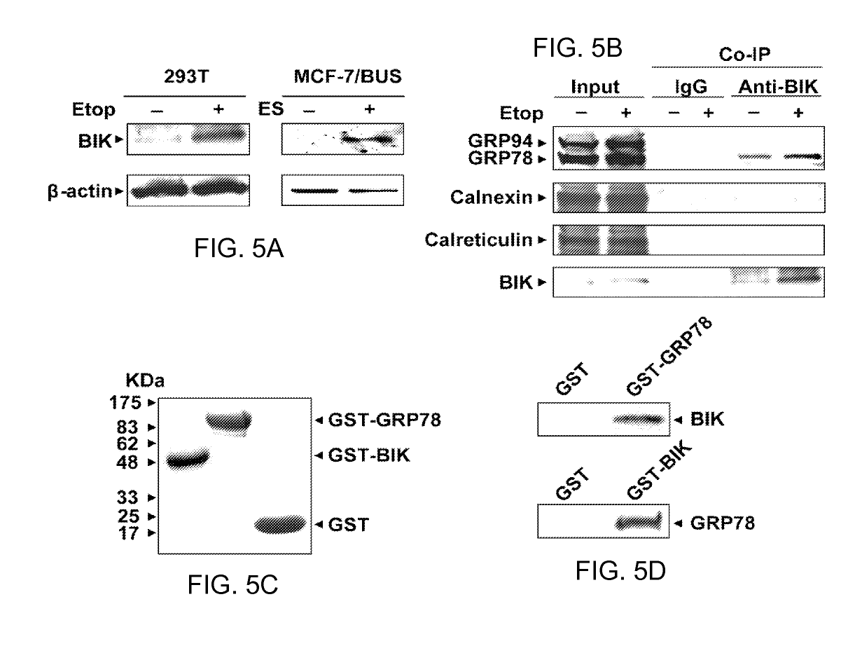

Endogenous BIK selectively forms complex with Grp78. The inducibility of BIK protein was determined by different stress conditions. In the human embryonic kidney cell line 293T, BIK protein was present at a low basal level under normal culture conditions. On treatment with etoposide, a topoisomerase I inhibitor, the level of BIK protein was substantially elevated (FIG. 5A). In the human breast carcinoma MCF-7/BUS cells, the level of BIK protein was dramatically induced by estrogen starvation (FIG. 5A). In contrast, ER stress inducers such as thapsigargin or tunicamycin do not induce BIK. Thus, the induction of BIK occurs under selective stress conditions in human cells.

As a first step toward understanding how BIK is regulated at the ER, the interactive partners of BIK were searched for using co-immunoprecipitation followed by Western blot with known ER proteins. BIK selectively interacts with Grp78. In coimmunoprecipitation assays, BIK complexed with Grp78 in both untreated cells and cells where BIK level was elevated by etoposide treatment (FIG. 5B). The interaction between endogenous Grp78 and BIK is specific because this complex was not observed using control IgG as the precipitating antibody, and other abundant ER proteins such as GRP94, calnexin, and calreticulin were not detected in the BIK immunoprecipitate (FIG. 5B). To confirm the physical interaction between Grp78 and BIK, they were both expressed as bacterial GST-fusion proteins. The yield and purity of the GST-proteins were confirmed by Coomassie blue staining (FIG. 5C). In pull-down assays, GST-Grp78, but not the GST protein, was able to bind BIK from total cell extract, and reversely, GST-BIK, but not the GST protein, was able to bind GRP78 (FIG. 5D). Thus, BIK and Grp78 form a complex both in vivo and in vitro.

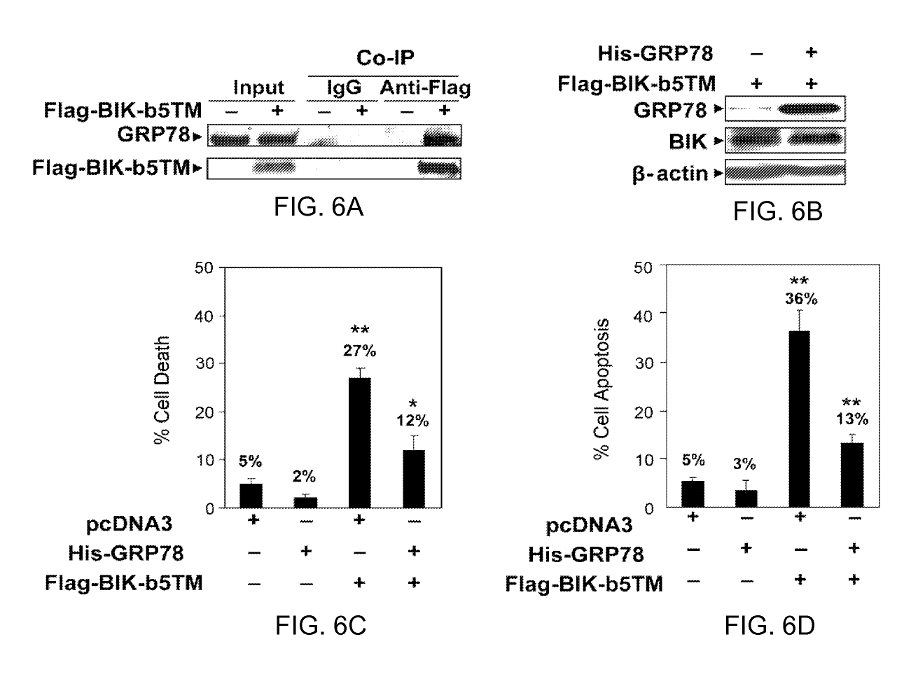

Grp78 binds ER-targeted BIK and blocks its apoptotic activity. To determine the functional interaction between Grp78 and BIK in the ER, 293T cells were transfected with a vector expressing Flag-tagged BIK, selectively targeted to the ER by using the cytochrome b5 transmembrane domain (b5TM). Western blot analysis confirmed expression of the Flag-tagged BIK-b5TM in the transfected cells and coimmunoprecipitation using anti-Flag antibody confirmed complex formation between Grp78 and the ER-targeted BIK in vivo (FIG. 6A). To test for the effects of Grp78 on BIK activity, the expression vector for ER-targeted BIK was cotransfected into 293T cells with either the expression vector for His-tagged Grp78 or the empty vector pcDNA3. Coexpression of the His-tagged Grp78 and Flag-tagged BIK in the transfected cells was confirmed by Western blot (FIG. 6B). Cell death determined by trypan blue exclusion reveals that cells expressing ER-targeted BIK exhibited a 5-fold increase in the percent of cell death compared with cells transfected with pcDNA3 (FIG. 6C). This increase was reduced by half in cells overexpressing Grp78, providing the first evidence that Grp78 is able to counteract cell death mediated by BIK. To determine whether the cell death observed was due to apoptosis, identical transfection experiments were done and the extent of apoptosis was determined by lipophilic cation fluorescent staining that detects changes in mitochondrial membrane potential. As summarized in FIG. 6D, ER-targeted BIK expression induced apoptosis in the transfected cells and Grp78 overexpression reduced ER-targeted BIK-induced apoptosis by 3-fold.

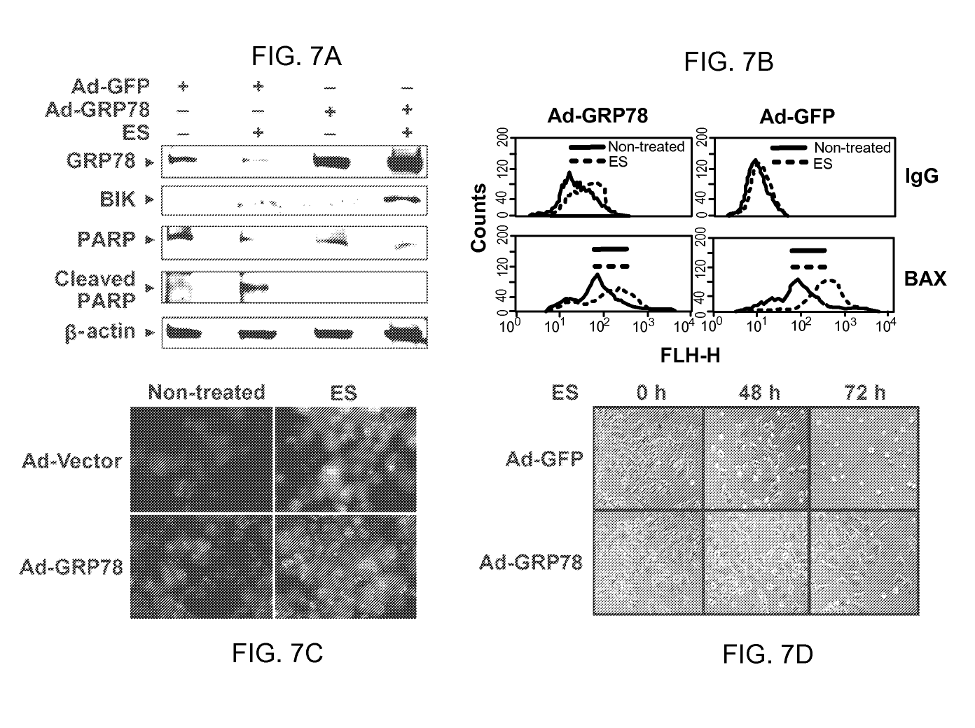

Grp78 overexpression inhibits estrogen starvation-induced BAX activation and apoptosis. Because BIK is an upstream regulator of BAX and estrogen starvation-induced apoptosis, inhibition of BIK activity by Grp78 overexpression should suppress these downstream pathways. To test this in the context of estrogen-dependent human cancer cells, MCF-7/BUS cells were infected with adenovirus vectors expressing either Grp78 (Ad-Grp78) or, as a control, GFP (Ad-GFP). Overexpression of Grp78 in the Ad-Grp78-infected cells was confirmed by Western blot (FIG. 7A). On estrogen starvation, BIK was induced, correlating with BAX activation (FIGS. 7A and 7B). Estrogen starvation resulted in fluorescent histogram curve shift with the mean fluorescence value increased from 77 to 313 when compared with the nontreated cells, indicating an increase of the active form of BAX as recognized by the BAX conformation specific antibody (FIG. 7B). In agreement with Grp78 counteracting BIK activity, the activation of BAX by estrogen starvation was suppressed in cells overexpressing Grp78 as compared with cells expressing GFP, with the mean fluorescence value decreased from 183 for cells expressing GFP to 70 for cells overexpressing Grp78 (˜48% suppression; FIG. 7B).

To test independently the protective effect of Grp78 in estrogen starvation-induced apoptosis, the same cells were subjected to the mitochondrial permeability transition assay. In this assay, the lipophilic MitoPT™ (Immunochemistry Technologies, LLC, Bloomington, Minn.) reagent penetrates the healthy mitochondria in nonapoptotic cells, aggregates, and produces red fluorescence in the negatively charged mitochondria. In early apoptotic cells, on collapse of the mitochondrial membrane potential, the MitoPT™ (Immunochemistry Technologies, LLC, Bloomington, Minn.) reagent distributes throughout the cell and fluoresces green. As shown in FIG. 7C, MCF-7/BUS cells overexpressing Grp78 showed substantial reduction in mitochondrial membrane potential change on 48 hours of estrogen starvation, as compared with cells infected with the empty vector. Further, because MCF-7/BUS cells are devoid of caspase-3, a useful indicator of apoptosis in these cells is estrogen starvation-induced cleavage of endogenous PARP. In non-apoptotic cells, PARP exists in its uncleaved form (116 kDa), whereas in apoptotic cells, PARP is cleaved by activated caspases into an 85-kDa fragment. As shown in FIG. 7A, the cleaved form of PARP was evident in estrogen-starved cells infected with Ad-GFP but was not observed in cells infected with Ad-Grp78. Finally, as shown by light microscopy, cells transfected with Ad-GFP gradually lost viability on estrogen starvation treatment, and by 72 hours, most cells exhibited rounded morphology, whereas ˜50% the Grp78 overexpressing cells were still viable (FIG. 7D). Collectively, these results provide several lines of evidence that Grp78 protects human breast cancer against estrogen starvation-induced apoptosis.

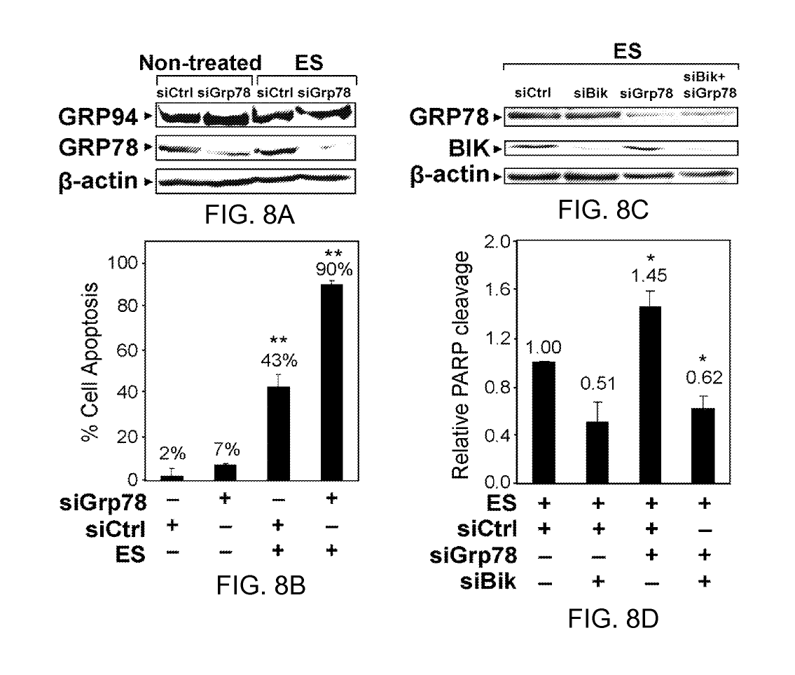

Knockdown of endogenous Grp78 sensitizes human breast cancer cells to estrogen starvation-induced apoptosis. To test directly whether the down-regulation of endogenous Grp78 protein level will sensitize human breast cancer to estrogen starvation-induced apoptosis, siRNA was used to knockdown expression of Grp78 in MCF-7/BUS cells. As shown in FIG. 8A, transient transfection of a Grp78-suppressing siRNA substantially reduced the level of Grp78 as compared with control siRNA. The siRNA against Grp78 is specific because it has no effect on the expression of another major ER chaperone protein, GRP94, or on the expression of h-actin. In cells growing in normal culture medium, siRNA against Grp78 and control siRNAs had little effect on the mitochondrial membrane potential (FIG. 8B). In contrast, in cells undergoing estrogen starvation for 24 hours, ere was a marked increase in apoptosis in cells transfected with the siRNA against Grp78 as compared with cells transfected with the control siRNA (FIG. 8B). Thus, Grp78 protects human breast cancer cells against estrogen starvation-induced apoptosis.

To test further whether this protective effect acts through BIK directly, siRNA was used to knock down Grp78 and BIK, either alone or in combination, in MCF-7/BUS cells subjected to estrogen starvation. To complement the measurement of apoptotic cells, the amount of apoptosis induced by estrogen starvation was determined by quantitation of PARP cleavage. As shown in FIG. 8C, the expression of Grp78 and BIK protein was substantially reduced by their specific siRNA as compared with control siRNA. Knockdown of BIK by siRNA decreased PARP cleavage as compared with cells transfected with control siRNA whereas knockdown of Grp78 increased PARP cleavage (FIG. 8D). Further, knockdown of BIK substantially reduced the enhanced PARP cleavage mediated by knockdown of Grp78 (FIG. 8D). The reduction was more than BIK knockdown alone. These results confirmed that BIK mediates estrogen starvation-induced apoptosis in MCF-7/BUS cells and further showed that Grp78 inhibits apoptosis in estrogen-starved breast cancer cells, in part, through suppression of BIK.

### Example 3

### GRP78 as a Predictor of Responsiveness to Chemotherapy

Materials and Methods

Study subjects. From 1989 to 1999, 432 female patients with stage II or III invasive breast cancer were treated in the University of Southern California (USC)/Norris Cancer Hospital (Los Angeles, Calif.), among whom 209 patients were treated with Adriamycin-based adjuvant chemotherapy. Demographic and clinical information were abstracted from hospital records. Tumor samples collected before the initiation of chemotherapy for 127 of the 209 patients were available for immunohistochemical staining. This study was approved by the USC Institutional Review Board (IRB). A waiver of informed consent was justified and granted by the IRB consistent with the waiver criteria of the common rule.

Immunohistochemical staining of Grp78 and evaluation. Five-micron sections of paraffin-embedded formalin fixed tissues were stained for Grp78 using anti-Grp78 H129 antibody (Santa Cruz Biotechnology, Santa Cruz, Calif.). Plasma cell staining was used as internal positive controls. The negative control was a sample within each batch, in which the primary antibody was omitted. Immunohistochemically stained slides from each subject were reviewed by a pathologist who was blinded to all clinical data. Staining was graded for intensity of staining (1, weak; 2, moderate; 3, strong) and percentage of cells stained (1, 0 to <10%; 2, 10 to <50%; 3, 50-100%). The overall index of Grp78 expression was determined based on the previous two variables: positive when both scores were 2 or above; negative otherwise. To examine the reader reproducibility of Grp78 immunohistochemistry evaluation, a random sample of 31 slides was chosen and reevaluated by the same pathologist, without knowledge of the previous results. The K coefficient was used to evaluate the agreement between two evaluations (Cohen, Educ. Psychol. Meas. 20:37-46 (1960)). The κ coefficient was 0.73 [95% confidence interval (95% CI), 0.50-0.98], indicating substantial agreement according to the Landis-Koch criterion (Landis and Koch, Biometrics 33:159-74 (1977)).

Statistical analyses. The measure of outcome, time to recurrence (TTR), was calculated from start of chemotherapy until the date of documented recurrence. For patients who had not experienced a recurrence at the time of last follow-up (death or last contact at the hospital or with the treating physician), TTR was censored at the date of last follow-up. Associations between demographic and clinical characteristics and GRP78 expression were evaluated using contingency tables and Pearson's χ² or Fisher's exact test. The association between TTR and Grp78 expression or other potential prognostic factors was evaluated using Kaplan-Meier plots and Cox Proportional hazards model (Kalbefleish and Prentice, The statistical analysis of failure time data. New York: John Wiley and Sons; 1980). All Ps reported are two sided and are based on the likelihood ratio test associated with the Cox model. Inspection of the hazards suggested that the assumption of constant proportional hazards was not well satisfied; analyses were repeated with the log-rank test and nearly identical hazard ratio (HR) estimates and Ps were obtained. For simplicity, all results are based on the Cox model.

To assess whether the association between Grp78 and TTR was independent of other prognostic factors, two approaches were used: (a) stratification by each prognostic factor and (b) stratification by quintiles of a propensity score. In post hoe examination, the relationship between Grp78 and TTR according to treatment modalities (types of chemotherapy, surgery, and radiation) was evaluated. The test of interaction was done by introducing an interaction term into the Cox model.

Results

Patient characteristics. In general, patients with tumor specimens available for Grp78 analysis were not substantially different in all major prognostic factors compared with those without available tumor specimens, with the exception that patients with available specimens were more likely to have undergone a mastectomy. When the associations between TTR and each of the patient and tumor characteristics were examined, as expected, tumor stage of T3 or T4, lymph node involvement, and high tumor grade were all associated with higher hazards of recurring. However, only the association with high tumor grade reached statistical significance at the 0.05 level (Table 6).

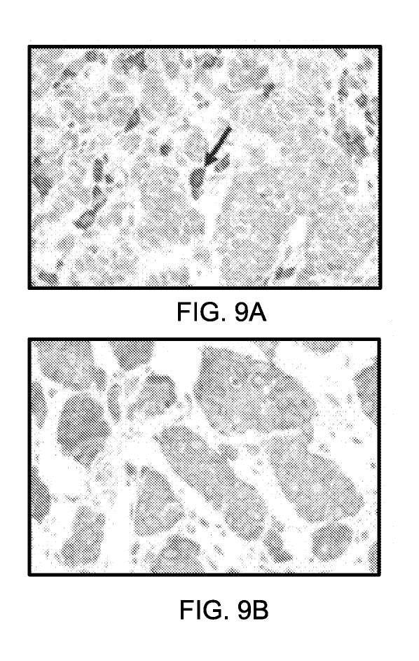

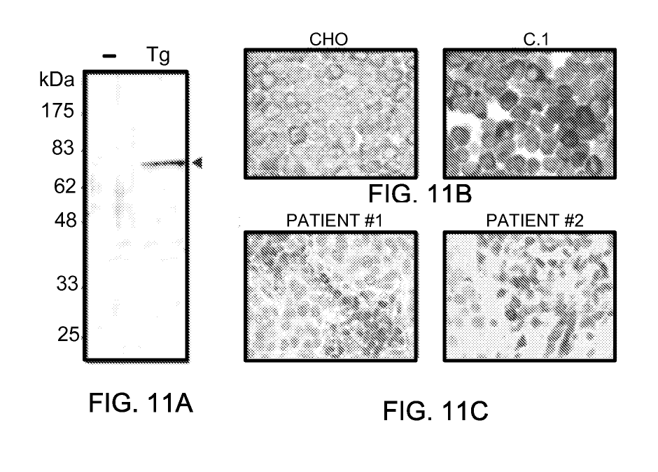

Grp78 expression in breast cancer patients. As an essential chaperone protein, Grp78 is expressed constitutively at varying basal levels in most cell types. For simplicity, tumors were classified into “Grp78-negative” or “Grp78-positive” groups based on the overall index of intensity of staining and the percentage of cells stained. Thus, the negative group included tumors that stained weakly and/or with limited stained areas, whereas positive tumors reached or exceeded the staining criterion. The specificity of the antibody against Grp78 was confirmed by Western blot of human cell lysates, as well as immunohistochemical staining of paraffin sections of established tissue culture cell lines that expressed differential level of Grp78 (FIGS. 11A to 11C). Further, plasma cells express high levels of Grp78, which facilitates immunoglobulin chain assembly. All subject samples contained plasma cells on their slides and their generally uniform high level immunoreactivity with the anti-Grp78 antibody conveniently served as internal positive controls (FIG. 11A to 11C). Representatives of Grp78-negative and Grp78-positive tumors are shown in FIG. 9. As expected for an endoplasmic reticulum protein, Grp78 staining was primarily in the perinuclear/cytoplasmic region. Among the 127 patients, 85 (67%) showed positive staining of Grp78, which was consistent across all subsets of patients, except subsets by tumor type (histology), where the numbers within categories were very small (Table 4).

TABLE 4
Associated between Grp78 expression and patient characteristics.

| | | | Grp78 |
|---|---|---|---|
| | Number of | Grp78 Expression | Expression |
| Patient Characteristics | Patients | % Positive¹ | P² |
| Total | 127 | 67 | |
| Menopausal status | | | |
| Premenopause | 66 | 64 | 0.41 |
| Postmenopause | 61 | 70 | |
| Histology | | | |
| Infiltrating ductal | 115 | 66 | 0.045 |
| carcinoma | | | |
| Infiltrating lobular | 10 | 90 | |
| carcinoma | | | |
| Others³ | 2 | 0 | |
| Stage | | | |
| T₁ (≦2 cm) | 44 | 68 | 0.94 |
| T₂ (>2, ≦5 cm) | 68 | 68 | |
| T₃, T₄ (>5 cm or | 11 | 73 | |
| inflammatory | | | |
| Tₓ (cannot be measured) | 4 | (25) | |
| Lymph node status | | | |
| Negative | 21 | 57 | 0.30 |
| Positive | 106 | 69 | |
| Lymphovascular invasion | | | |
| Negative | 77 | 66 | 0.84 |
| Positive | 50 | 68 | |
| ER/PR status⁴ | | | |
| −/− | 27 | 63 | 0.62 |
| −/+, +/−, or +/+ | 97 | 68 | |
| Unknown | 3 | (66) | |
| HER-2/neu status | | | |
| Negative | 76 | 66 | 0.96 |
| Positive | 23 | 65 | |
| Unknown | 28 | (71) | |
| Grade⁵ | | | |
| 1 + 2 | 52 | 65 | 0.84 |
| 3 | 52 | 67 | |
| Unknown | 11 | (64) | |
| Chemotherapy | | | |
| Adriamycin based⁶ | 102 | 67 | 0.90 |
| Taxanes added⁷ | 25 | 68 | |
| Surgery type and radiation | | | |
| therapy | | | |
| Segmental, radiated | 34 | 62 | 0.45 |
| Segmental, not radiated | 1 | 0 | |
| Mastectomy, radiated | 22 | 73 | |
| Mastectomy, not | 70 | 68 | |
| radiated | | | |

¹Percent of subjects with GRP78-positive staining.
²Based on χ² test, except for histology and surgery type and radiation therapy, for which P is based on Fisher's exact test. Excludes patients with unknown status.
³Others include medullary carcinoma and papillary carcinoma.
⁴Estrogen receptor/progesterone receptor status.
⁵Limited to infiltrating ductal carcinoma.
⁶Adriamycin with one or more of cyclophosphamide, 5-fluorouracil, or methotrexate.
⁷Adriamycin-based chemotherapy followed by or combined with taxanes.

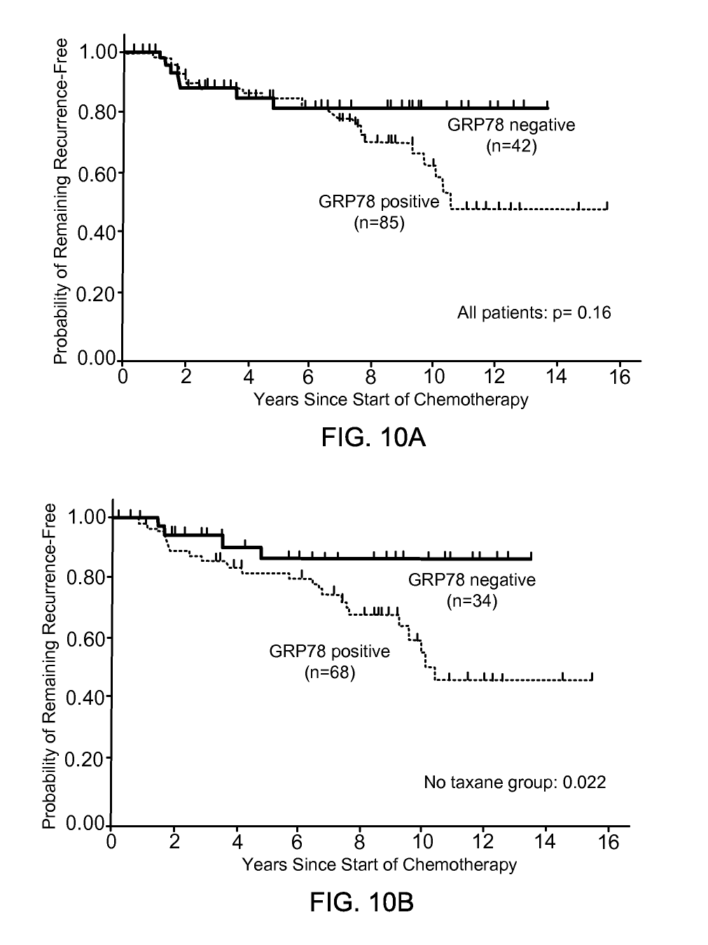

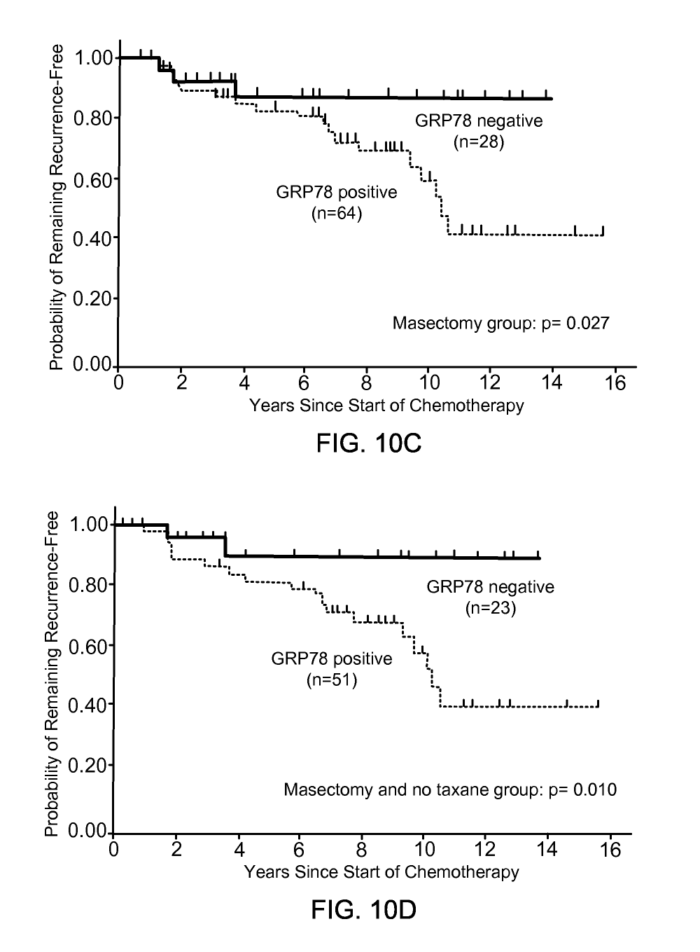

Association between Grp78 and TTR. Among the 127 study subjects who received Adriamycin-based therapy, the Grp78-positive group showed an increased likelihood of recurring (HR, 1.78; 95% CI, 0.77-4.14; FIG. 10A; Table 5).

TABLE 5
Relative risk of recurrence associated with Grp78 expression.

| | | | | Univariate | | Multivariable | |
|---|---|---|---|---|---|---|---|
| | | | | analysis | | analysis | |
| Treatment | | | | HR (95% | | HR (95% | |
| Characteristics | Grp78 | n | Events¹ | CI) | P² | CI) | P² |
| Overall | | | | | | | |
| analysis | | | | | | | |
| Total subjects | Negative | 42 | 7 | 1 | | 1 | |
| | Positive | 85 | 24 | 1.78 (0.77-4.14) | 0.16 | 1.76 (0.74-4.17) | 0.18 |
| Subgroup | | | | | | | |
| analyses | | | | | | | |
| Chemotherapy | | | | | | | |
| Adriamycin | Negative | 34 | 4 | 1 | | 1 | |
| based³ | | | | | | | |
| | Positive | 68 | 23 | 3.00 (1.04-8.70) | 0.022 | 3.00 (1.02-8.84) | 0.026 |
| Taxanes added⁴ | Negative | 8 | 3 | 1 | | 1 | |
| | Positive | 17 | 1 | 0.15 (0.016-1.46) | 0.072 | 0.24 (0.020-3.00) | 0.24 |
| P for | | | | | 0.012 | | 0.012 |
| interaction | | | | | | | |
| (GRP78 and | | | | | | | |
| chemotherapy) | | | | | | | |
| Surgery type | | | | | | | |
| Segmental | Negative | 14 | 4 | 1 | | 1 | |
| mastectomy | | | | | | | |
| | Positive | 21 | 5 | 0.74 (0.20-2.77) | 0.66 | 0.53 (0.11-2.54) | 0.42 |
| Mastectomy | Negative | 28 | 3 | 1 | | 1 | |
| | Positive | 64 | 19 | 3.33 (0.98-11.30) | 0.027 | 2.53 (0.73-8.75) | 0.11 |
| P for | | | | | 0.078 | | 0.28 |
| interaction | | | | | | | |
| (GRP78 and | | | | | | | |
| Surgery) | | | | | | | |

¹Number of recurrences.
²Stratified analysis using propensity score (based on tumor stage, lymph node status, and grade) divided into quintiles.
³ Ps from likelihood ratio test based on Cox model.
⁴Adriamycin with one or more cyclophosphamide, 5-fluorouracil, or methotrexate.
⁵Adriamycin-based chemotherapy followed by or combined with taxanes.

Although this trend does not achieve statistical significance (P=0.16), the observed HR is very close to the value stipulated in the design (1.70 or 70% increase). Adjustment for each patient and tumor characteristic did not substantially change the results (Table 7). In a multivariable analysis, the magnitude of the association remained the same even after adjusting for tumor stage, lymph node status, and grade using the propensity score.

Post hoc analyses of Grp78 staining and TTR in subsets of patients by the treatment modalities revealed two strong and interesting trends (Table 5). First, the HR for the Grp78-positive group increased significantly among patients treated with Adriamycin-based chemotherapy who did not receive further treatment with taxane (paclitaxel or docetaxel; HR, 3.00; P=0.022; also see FIG. 10B). The interaction (differences in the two HRs depending on addition of taxanes) was statistically significant (P=0.012). Further, the adjustment for tumor stage, lymph node status, and grade (using propensity scores) did not change the results. In agreement with taxane treatment exerting an opposing trend, among patients treated with Adriamycin combined with or followed by a taxane, positive Grp78 seemed to have a lower risk of recurrence with borderline significance (HR, 0.15; P=0.072).

Second, when stratified by type of surgery (segmental mastectomy versus mastectomy), a positive association between Grp78 expression and TTR was observed among patients who underwent mastectomy (HR, 3.33; P=0.027; also see FIG. 10C). The interaction between Grp78 expression and surgery type with regard to TTR was borderline significant (P=0.078). However, after adjustment for tumor stage, lymph node status, and grade (using propensity scores), the interaction was not statistically significant (P=0.28), and the association among patients with mastectomy was reduced by 25% (HR, 2.53; P=0.11). When evaluating the patients who had mastectomy and did not receive a taxane, the association between positive Grp78 and TTR was stronger (HR, 4.82; 95% CI, 1.12-20.87; P=0.010; FIG. 10D) and remained statistically significant after adjustment for tumor stage, lymph node status, and grade (HR, 3.77; 95% CI, 0.85-16.66; P=0.041). Most patients who had mastectomy did not receive radiation therapy, whereas all but one patient who had segmental mastectomy received radiation therapy. Stratification by radiation therapy yielded similar results as with stratification by type of surgery.

Patient Characteristics. Study design. Based on the assumption that approximately 65% of patients will be classified as overexpressing Grp78 (Fernandez et al., Breast Cancer Res. Treat. 59:15-26 (2000)), this study was designed to include 300 female patients with stage II or III invasive breast cancer who had received adriamycin-based adjuvant chemotherapy during 1999 or earlier, to allow for five years of minimum follow-up. The target of 300 patients, with at least five years of follow-up, was determined to ensure 80% power to detect a difference in time to recurrence (TTR) according to Grp78 expression level, if GRP78 positively conferred a 70% increase in the recurrence rate as estimated by the hazard ratio (HR). Since adriamycin-based chemotherapy was not prescribed routinely to patients with stage II or III breast cancer prior to 1989, patients receiving this treatment prior to 1989 were not included to avoid any bias due to patient selection.

Statistical analysis. The following prognostic variables (covariates) were considered: age at diagnosis (<40, 40-49, 50-59, 60+), menopausal status (premenopausal, postmenopausal), histology (infiltrating ductal carcinoma and infiltrating lobular carcinoma, and others including medullary carcinoma and papillary carcinoma), T stage (T1, T2, T3/T4, unknown) and lymph node status (positive, negative), grade (1 or 2, 3, unknown or not-applicable), lymphovascular invasion (yes, no), extranodal extension (yes, no), estrogen receptor (ER) and progesterone receptor (PR) (positive, negative), surgery type (mastectomy, segmental mastectomy), radiation therapy (yes, no), and tamoxifen treatment (yes, no). Menopausal status at diagnosis had been self-reported in original medical records. If a woman had bilateral oophorectomy prior to diagnosis, she was classified as postmenopausal. For women with hysterectomy other than bilateral oophorectomy, their age at diagnosis was considered to classify their menopausal status (age <50: premenopausal, age ≧50: postmenopausal). Perimenopausal women were classified as premenopausal (n=5). The above age cutpoint was applied to classify menopausal status of women with unknown menopausal status (n=4). Some patients were treated with tamoxifen for a brief period rather than the recommended 5 year period. Patients who were on tamoxifen less than or equal to 3 months were classified as ‘not treated.’

Given the size of the study and the number of prognostic variables, instead of attempting to assess the association between Grp78 and TTR controlling for all these covariates simultaneously, two strategies were used to assess whether the association between Grp78 status and TTR was dependent of the standard prognostic variables. First, the association was reexamined after stratifying by each of the individual prognostic variables—separately. Second, a propensity score was calculated based on T stage, lymph node status, and grade—variables with the largest (or smallest) hazard ratios, when examined singly. The association between Grp78 and TTR was re-evaluated after stratifying by the propensity score divided into quintiles. The propensity score is a method to adjust simultaneously for 2+ observed covariates (Joffe et al., Am. J. Epidemiol. 150:327-33 (1999)).

TABLE 6
Association of patient characteristics with tumor block availability or
time to recurrence.

| | Association with | | | | |
|---|---|---|---|---|---|
| | Tumor | | | Association | |
| | Block | | | with Time | |
| Patient | Availability | | | to Recurrence¹ | |
| Characteristics | No (%) | Yes (%) | p-value² | Hazard Ratio | p-value³ |
| Total | 82 (100) | 127 (100) | | | |
| Menopausal status | | | | | |
| Premenopause | 42 (51) | 66 (52) | 0.92 | 1 | |
| Postmenopause | 40 (49) | 61 (48) | | 1.53 | 0.24 |
| Histology | | | | | |
| Infiltrating | 69 (84) | 115 (90) | 0.12 | 1 | 0.48 |
| ductal | | | | | |
| carcinoma | | | | | |
| Infiltrating | 7 (8) | 10 (8) | | 1.58 | |
| lobular | | | | | |
| carcinoma | | | | | |
| Others⁴ | 6 (7) | 2 (2) | | — | |
| Stage | | | | | |
| T₁ (≦2 cm) | 26 (32) | 44 (35) | 0.69 | 1 | 0.59 |
| T₂ (>2, ≦5 cm) | 46 (56) | 68 (54) | | 1.08 | |
| T₃, T₄ (>5 cm or | 10 (12) | 11 (9) | | 1.78 | |
| inflammatory) | | | | | |
| Tₓ (cannot be | 0 (0) | 4 (3) | | — | |
| measured) | | | | | |
| Lymph node | | | | | |
| status | | | | | |
| Negative | 20 (24) | 21 (17) | 0.16 | 1 | 0.28 |
| Positive | 62 (76) | 106 (83) | | 1.82 | |
| Lymphovascular | | | | | |
| invasion | | | | | |
| Negative | 45 (55) | 77 (61) | 0.47 | 1 | 0.67 |
| Positive | 36 (44) | 50 (39) | | 0.85 | |
| Unknown | 1 (1) | 0 (0) | | — | |
| ER/PR status⁵ | | | | | |
| −/− | 23 (28) | 27 (21) | 0.20 | 1 | 0.96 |
| −/+, +/−, or +/+ | 54 (66) | 97 (76) | | 1.02 | |
| Unknown | 5 (6) | 3 (2) | | — | |
| HER-2/neu status | | | | | |
| Negative | 28 (34) | 76 (60) | 0.70 | 1 | 0.79 |
| Positive | 10 (12) | 23 (18) | | 0.86 | |
| Unknown | 44 (54) | 28 (22) | | — | |
| Grade⁶ | | | | | |
| 1 + 2 | 29 (42) | 52 (45) | 0.92 | 1 | 0.005 |
| 3 | 30 (43) | 52 (45) | | 3.19 | |
| Unknown | 10 (14) | 11 (10) | | — | |
| Chemotherapy | | | | | |
| Adriamycin | 67 (82) | 102 (80) | 0.80 | 1 | 0.86 |
| based⁷ | | | | | |
| Taxanes added⁸ | 15 (18) | 25 (20) | | 1.11 | |
| Surgery type and | | | | | |
| radiation therapy | | | | | |
| Segmental, | 35 (43) | 34 (27) | 0.002 | 1 | 0.43 |
| radiated | | | | | |
| Segmental, not | 5 (6) | 1 (1) | | — | |
| radiated | | | | | |
| Mastectomy, | 15 (18) | 22 (17) | | 1.55 | |
| radiated | | | | | |
| Mastectomy, | 27 (33) | 70 (55) | | 0.85 | |
| not radiated | | | | | |

¹Among patients with tumor blocks (n = 127).
²Based on χ² test, except for histology and surgery type and radiation therapy, for which p-value is based on Fisher's exact test.
³p-values from likelihood ratio test based on Cox model.
⁴Others include medullary carcinoma and papillary carcinoma, for tumor block available and not-available group. Tumor-block non-available group also includes muscinous carcinoma and a typical medullary carcinoma.
⁵Estrogen receptor/progesterone receptor status.
⁶Limited to infiltrating ductal carcinoma.
⁷Adriamycin with one or more of cyclophosphamide, 5-fluorouracil, or methotrexate.
⁸Adriamycin-based chemotherapy followed by or combined with taxanes.

TABLE 7
Multivariable analyses stratified by each of the covariates.

| | Hazard Ratio for Subset of Patients | | | | | |
|---|---|---|---|---|---|---|
| | | | | | | Adriamycin- |
| | | Adriamycin- | | | | based |
| | | based | | | | (without |
| | | (without | Taxanes | Segmental | | taxanes)¹, |
| | All | taxanes)¹ | added² | mastectomy | Mastectomy | mastectomy |
| Number of | 127 | 102 | 25 | 35 | 92 | 74 |
| patients | | | | | | |
| Univariate | 1.78 | 3.00 | 0.15 | 0.74 | 3.33 | 4.82 |
| analysis | | | | | | |
| Stratified | | | | | | |
| variables³ for | | | | | | |
| multivariable | | | | | | |
| analysis | | | | | | |
| Menopausal status | 1.73 | 2.89 | 0.078 | 0.73 | 3.13 | 4.37 |
| Age at diagnosis | 1.55 | 2.54 | 0.25 | 0.57 | 2.78 | 3.82 |
| Race | 1.63 | 2.46 | 0.23 | 0.69 | 2.46 | 3.44 |
| T stage | 1.95 | 2.96 | 0.25 | 1.00 | 2.96 | 4.46 |
| Lymph node | 1.73 | 2.85 | 0.17 | 0.73 | 3.08 | 4.39 |
| status | | | | | | |
| Grade⁴ | 1.71 | 2.85 | 0.16 | 0.70 | 3.23 | 4.55 |
| Extranodal | 1.84 | 3.06 | 0.11 | 0.78 | 3.42 | 4.98 |
| extension | | | | | | |
| Lymphovascular | 1.74 | 2.80 | 0.14 | 0.76 | 3.46 | 4.81 |
| invasion | | | | | | |
| ER/PR status⁴ | 1.91 | 3.14 | 0.19 | 0.75 | 3.36 | 4.92 |
| Her-2/neu⁴ | 1.80 | 3.08 | 0.17 | 0.69 | 3.47 | 4.99 |
| Surgery type | 1.87 | 3.11 | 0.16 | — | — | — |
| Radiation | 1.84 | 3.12 | 0.14 | 0.95 | 3.43 | 4.83 |
| Chemotherapy | 1.76 | — | — | 0.71 | 3.31 | — |
| (whether taxanes | | | | | | |
| added) | | | | | | |
| Tamoxifen | 1.78 | 2.97 | 0.18 | 0.80 | 3.28 | 4.76 |

¹Adriamycin with one or more of cyclophosphamide, 5-fluorouracil or methotrexate - without taxanes.
²Adriamycin-based chemotherapy followed by or combined with taxanes.
³Categorization of each covariate is described in the text for supplemental data in detail.
⁴Patients with unknown or unavailable grade or Her-2/neu or ER/PR status were included in the analysis as a separate category. ER/PR: estrogen receptor/progesterone receptor.

### Example 4

### Bioinformatics Analysis of Various Cell Lines

As shown in Table 8, the cell lines with high levels of Grp78 were listed under “high probability” and the cell lines with low levels of Grp78 were listed under “low probability.”

TABLE 8
Bioinformatic analysis.

| High Probability | Low Probability |
|---|---|
| Leukemia Promyelocytic (HL-60) | Skeletal Muscle |
| Leukemia Chronic Meylogenous (K562) | Cerebral Cortex |
| Leukemia Lymphoblastic (MOLT-4) | Lung |
| Liver (fetal) | Prostate |
| Thyroid | Heart |

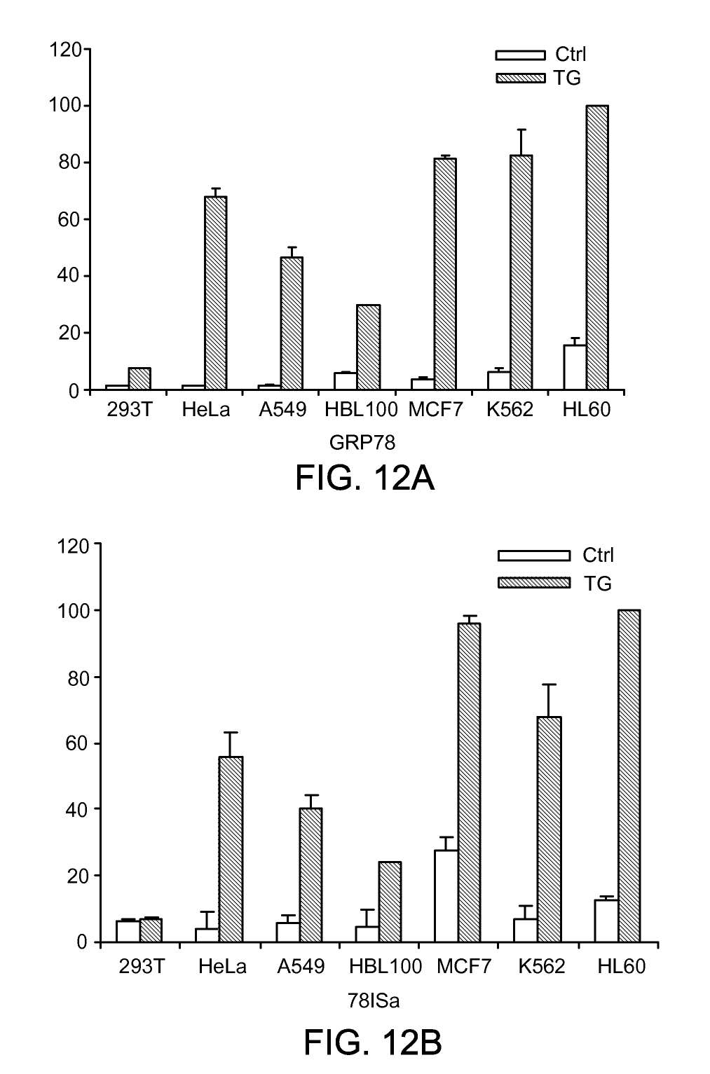

FIGS. 12A and 12B are graphs of Q-PCR analysis of various cell lines under normal conditions (control, open bars) and following exposure to thapsigargin (TG, striped bars). FIG. 12A shows the levels of Grp78 mRNA. FIG. 12B shows the levels of the mRNA splice variant of Grp78 (78ISa).

What is claimed is:

1. A method of determining whether a subject with cancer is at risk for developing resistance to hormonal therapy comprising:
a) selecting a subject at risk for developing resistance to hormonal therapy;
b) obtaining a biological sample from the subject; and
c) determining the level of expression of GRP78 in the biological sample, wherein overexpression of GRP78 in the biological sample as compared to a control indicates that the subject is at risk for developing resistance to hormonal therapy.

2. The method of claim 1, wherein the cancer is prostate cancer.

3. The method of claim 2, wherein the prostate cancer is androgen dependent.

4. The method of claim 3, wherein the hormonal therapy is an anti-androgen agent.

5. The method of claim 3, wherein the hormonal therapy is finasteride.

6. The method of claim 1, wherein the cancer is breast cancer.

7. The method of claim 6, wherein the breast cancer is hormone receptor positive breast cancer.

8. The method of claim 7, wherein the hormonal therapy is an anti-estrogen agent.

9. The method of claim 7, wherein the hormonal therapy is an aromatase inhibitor or tamoxifen.

10. The method of claim 9, wherein the aromatase inhibitor is selected from the group consisting of exemestane, aminoglutethimide, 4-androstene-3,6,17-trione, anastrozole and letrozole.

11. The method of claim 1, wherein the level of expression of GRP78 is determined by a method selected from the group consisting of RT-PCR, Northern blot, Western blot and ELISA.

12. A method for treating castration resistant prostate cancer in a subject comprising:
a) selecting a subject at risk for developing resistance to hormonal therapy; and b) contacting the castration resistant prostate cancer cells in the subject with one or more agents that inhibit expression or activity of GRP78 and a therapeutic agent.

13. The method of claim 12, wherein expression of GRP78 mRNA or GRP78 protein is inhibited.

14. The method of claim 12, wherein the activity of GRP78 is inhibited.

15. The method of claim 12, wherein the GRP78 gene or its promoter is inactivated.

16. The method of claim 12, wherein the agent that inhibits expression of GRP78 is selected from the group consisting of an antisense molecule, a triple helix molecule, a ribozyme and an siRNA.

17. The method of claim 12, wherein the agent that inhibits activity of GRP78 is a GRP78 antagonist.

18. The method of claim 17, wherein the GRP78 antagonist is selected from the group consisting of an antibody to GRP78, (−)-epigallocatechin gallate and genistein.

19. The method of claim 17, wherein the GRP78 antagonist is a combination of a taxane and doxirubicin.

20. The method of claim 19, wherein the taxane is paclitaxel or docetaxel.

21. The method of claim 12, wherein the therapeutic agent is an anti-hormonal agent or a chemotherapeutic agent.

22. The method of claim 12, wherein the therapeutic agent is an anti-androgen agent.

23. The method of claim 12, wherein the therapeutic agent is finasteride.

24. The method of claim 17, wherein the GRP78 antagonist is not a combination of a taxane and doxirubicin.

25. A method of treating hormone receptor positive breast cancer in a subject comprising:
a) selecting a subject at risk for developing resistance to hormonal therapy; and b) contacting the hormone receptor positive breast cancer cells in the subject with one or more agents that inhibit expression or activity of GRP78 and a therapeutic agent.

26. The method of claim 25, wherein expression of GRP78 mRNA or GRP78 protein is inhibited.

27. The method of claim 25, wherein the activity of GRP78 is inhibited.

28. The method of claim 25, wherein the GRP78 gene or its promoter is inactivated.

29. The method of claim 25, wherein the agent that inhibits expression of GRP78 is selected from the group consisting of an antisense molecule, a triple helix molecule, a ribozyme and an siRNA.

30. The method of claim 25, wherein the agent that inhibits activity of GRP78 is a GRP78 antagonist.

31. The method of claim 30, wherein the GRP78 antagonist is selected from the group consisting of an antibody to GRP78, (−)-epigallocatechin gallate and genistein.

32. The method of claim 30, wherein the GRP78 antagonist is a combination of a taxane and doxirubicin.

33. The method of claim 32, wherein the taxane is paclitaxel or docetaxel.

34. The method of claim 25, wherein the therapeutic agent is an anti-hormonal agent or a chemotherapeutic agent.

35. The method of claim 34, wherein the anti-hormonal agent is an anti-estrogen agent.

36. The method of claim 25, wherein the therapeutic agent is an aromatase inhibitor or tamoxifen.

37. The method of claim 36, wherein the aromatase inhibitor is selected from the group consisting of exemestane, aminoglutethimide, 4-androstene-3,6,17-trione, anastrozole and letrozole.

38. The method of claim 30, wherein the GRP78 antagonist is not a combination of a taxane and doxirubicin.

39. A method of determining whether a subject with cancer is at risk for developing resistance to a chemotherapeutic agent comprising:
a) selecting a subject at risk for developing resistance to a chemotherapeutic agent;
b) obtaining a biological sample from the subject; and
c) determining the level of expression of GRP78 in the biological sample, wherein overexpression of GRP78 in the biological sample as compared to a control indicates that the subject is at risk for developing resistance to the chemotherapeutic agent.

40. The method of claim 39, wherein the cancer is breast cancer.

41. The method of claim 39, wherein the chemotherapeutic agent is a topoisomerase inhibitor.

42. The method of claim 41, wherein the topoisomerase inhibitor is doxorubicin.

43. The method of claim 39, wherein the level of expression of GRP78 is determined by a method selected from the group consisting of RT-PCR, Northern blot, Western blot and ELISA.

44. A method of treating breast cancer in a subject comprising:
a) selecting a subject at risk for developing resistance to a chemotherapeutic agent; and
b) contacting the breast cancer cells in the subject with one or more agents that inhibits expression or activity of GRP78 and a chemotherapeutic agent.

45. The method of claim 44, wherein the chemotherapeutic agent is a topoisomerase inhibitor.

46. The method of claim 45, wherein the topoisomerase inhibitor is doxorubicin.

47. The method of claim 44, wherein the agent that inhibits expression of GRP78 is selected from the group consisting of an antisense molecule, a triple helix molecule, a ribozyme and an siRNA.

48. The method of claim 44, wherein the agent that inhibits activity of GRP78 is a GRP78 antagonist.

49. The method of claim 48, wherein the GRP78 antagonist is selected from the group consisting of an antibody to GRP78, (−)-epigallocatechin gallate and genistein.

50. The method of claim 48, wherein the GRP78 antagonist is a combination of a taxane and doxirubicin.

51. The method of claim 50, wherein the taxane is paclitaxel or docetaxel.

52. The method of claim 48, wherein the GRP78 antagonist is not a combination of a taxane and doxirubicin.


SEQUENCE LISTING (2 sequences)

SEQ ID NO: 1 - DNA, 20 bp, Artificial Sequence
  Synthetically generated siRNA
gagcgcauugauacuagann

SEQ ID NO: 2 - RNA, 21 nt, Artificial Sequence
  Synthetically generated siRNA
aagaccccucuccagagacau